SEQUENCE LISTING (13 sequences)

SEQ ID NO: 1 - DNA, 2759 bp, Artificial
  Plasmid pMT - ctxBthyA-2
ctcgaggtttgttcctgattggttacggcgcgtttcgcatcattgttgagtttttccgccagcccgacgcgcagtttaccggtgcctgggtgcagtacatcagcatggggcaaattctttccatcccgatgattgtcgcgggtgtgatcatgatggtctgggcatatcgtcgcagcccacagcaacacgtttcctgaggaaccatgaaacagtatttagaactgatgcaaaaagtgctcgacgaaggcacacagaaaaacgaccgtaccggaaccggaacgctttccatttttggtcatcagatgcgttttaacctgcaagatggattccggctggtgacaactaaacgttgccacctgcgttccatcatccatgaactgctgtggtttctgcagggcgacactaacattgcttatctacacgaaaacaatgtcaccatctgggacgaatgggccgatgaaaacggcgacctcgggccagtgtatggtaaacagtggcgcgcctggccaacgccagatggtcgtcatattgaccagatcactacggtactgaaccagctgaaaaacgacccggattcgcgccgcattattgtttcagcgtggaacgtaggcgaactggataaaatggcgctggcaccgtgccatgcattcttccagttctatgtggcagacggcaaactctcttgccagctttatcagcgctcctgtgacgtcttcctcggcctgccgttcaacattgccagctacgcgttattggtgcatatgatggcgcagcagtgcgatctggaagtgggtgattttgtctggaccggtggcgacacgcatctgtacagcaaccatatggatcaaactcatctgcaattaagccgcgaaccgcgtccgctgccgaagttgattatcaaacgtaaacccgaatccatcttcgactaccgtttcgaagactttgagattgaaggctacgatccgcatccgggcattaaagcgccggtggctatctaattacgaaacatcctgccagagccgacgccagtgtgcgtcggtttttttaccctccgttaaattcttcgagacgccttcccgaaattttgcaacgtcctgcaacggcgtaaatagtccggaagatgcgccgaagaaatagaaacgtcgaatcaagcttatcgataccgtcgaccttgaagagcaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggcatctttatagtcctgtcgggtttcgccacctctgacttgatcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgatggaagagcagatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattcgggatgaattatgaataaagtaaaattttatgttttatttacggcgttactatcctctctatgtgcacacggagctcctcaaaatattactgatttgtgtgcagaataccacaacacacaaatacatacgctaaatgataagatattttcgtatacagaatctctagctggaaaaagagagatggctatcattacttttaagaatggtgcaacttttcaagtagaagtaccaggtagtcaacatatagattcacaaaaaaaagcgattgaaaggatgaaggataccctgaggattgcatatcttactgaagctaaagtcgaaaagttatgtgtatggaataataaaacgcctcatgcgattgccgcaattagtatggcaaattaaactagtcaattgaagcttagcccgcctaatgagcgggctttttttt

SEQ ID NO: 2 - protein, 21 aa, Artificial
  Escherichia coli
MNKVKFYVLFTALLSSLCAHG

SEQ ID NO: 3 - protein, 6 aa, Artificial
  Vibrio cholerae
TPQNIT

SEQ ID NO: 4 - protein, 6 aa, Artificial
  Vibrio cholerae
APQNIT

SEQ ID NO: 5 - protein, 21 aa, Artificial
  Escherichia coli
MNKVKCYVLFTALLSSLCAYG

SEQ ID NO: 6 - protein, 28 aa, Artificial
  Vibrio cholerae
MNKVKFYVLFTALLSSLCAHGAPGYAHG

SEQ ID NO: 7 - protein, 15 aa, Artificial
  Vibrio cholerae
NKVKFYVLFTALLSS

SEQ ID NO: 8 - DNA, 31 bp, Artificial
  Vibrio cholerae
gctctagagctacggtcttgatttacggtat

SEQ ID NO: 9 - DNA, 33 bp, Artificial
  Escherichia coli
gggggctcgaggtttgttcctgattggttacgg

SEQ ID NO: 10 - DNA, 36 bp, Artificial
  Escherichia coli
ggggggtcgacgtttctatttcttcggcgcatcttc

SEQ ID NO: 11 - DNA, 40 bp, Artificial
  Vibrio cholerae
gggggactagtttaatttgccatactaattgcggcaatcg

SEQ ID NO: 12 - DNA, 41 bp, Artificial
  Vibrio cholerae
gggggactagtcaattgaagcttaagcccgcctaatgagcg

SEQ ID NO: 13 - protein, 21 aa, Artificial
  Escherichia coli
MNKVKCYVLFTALLSSLCAYG

(19) United States
(12) Patent Application Publication
(10) Pub. No.: US 2007/0212757 A1
(43) Pub. Date: Sep. 13, 2007

(54) Expression System for the B Subunit of Cholera Toxin

(75) Inventors: Nils Carlin, Hasselby (SE); Michael Lebens, Goteborg (SE)

Correspondence Address: BACON & THOMAS, PLLC, 625 SLATERS LANE, FOURTH FLOOR, ALEXANDRIA, VA, 22314, US

(73) Assignee: SBL Vaccin AB, Stockholm (SE)

(21) Appl. No.: 10/577,861

(22) PCT Filed: Oct. 29, 2004
(86) PCT No.: PCT/SE04/01571

§ 371 Date: Nov. 1, 2006

(30) Foreign Application Priority Data
Oct. 31, 2003 (GB) 0325494.3

(51) Int. Cl. C12P 21/06; C12N 1/21; C12N 15/74; C07H 21/04; C07K 14/28

(52) U.S. Cl. 435/69.3; 435/252.3; 435/471; 530/350; 536/23.7

(57) ABSTRACT

The present invention provides an expression system for producing a B subunit of a cholera toxin (CTB) wherein the expression system comprises a Vibrio cholerae host cell lacking the functionality of a thyA gene; and an expression vector comprising a functional thyA gene and a CTB gene which is substantially free of the flanking sequences immediately contiguous by the 5′ and 3′ end of the CTB gene in the naturally occurring genome of the host cell from which the CTB gene is derived. The present invention also provides a method of producing CTB, and an isolated nucleic acid construct that is used as an expression vector in the expression system.

The present invention relates to an expression system for producing the B subunit of cholera toxin (CTB), a method of producing CTB and an isolated nucleic acid construct as an expression vector for use in the expression system,

### BACKGROUND

The non-toxic B subunit of cholera toxin (CTB) is an effective oral immunizing agent, which in a large field trial, has been shown to afford protection against both cholera and enterotoxigenic E. coli caused diarrhea (Sanchez and Holmgren 1989 PNAS 86: 481-485). This has made CTB, as such, an important component, together with killed whole V. cholerae cells, of an oral cholera vaccine. Moreover, CTB has attracted much interest recently as an immunogenic carrier for various other peptides or carbohydrate antigens and as an immunomodulator for down regulating the immune response. These findings have emphasised the need to increase the yields of CTB for large scale production to facilitate, in part, vaccine development based on the use of CTB.

The choice of expression system for producing CTB depends on many factors, including the proteolytic stability of the protein, whether or not the protein is secretable and the acceptable costs of the final CTB product. There are four major expression systems which are commonly used to produce vaccine antigens. These are bacterial, yeast, insect and mammalian expression systems. In addition, transgenic plant expression systems have started to emerge with the aim of utilising the plant both for production of the subunit vaccine and for vaccine delivery via the edible plant. By way of example, WO 99/54452 discloses chimeric gene constructs comprising a CTB coding sequence and an autoantigen coding sequence, plant cells and transgenic plants transformed with said chimeric gene constructs, and methods of preparing an edible vaccine from these plant cells and transgenic plants

The expression of recombinant genes in bacterial host cells is most often achieved by the introduction of episomal self-replicating elements (such as plasmids) that encode the structural gene of the protein of interest under the control of an appropriate promoter, into host bacteria. Such plasmids are most commonly maintained by the inclusion of selective marker genes that encode proteins that confer resistance to specific antibiotics (such as ampicillin, chloramphenicol, kanamycin, tetracycline and the like). The plasmids are then maintained in the host by addition of the appropriate antibiotic to the culture medium.

Whilst E. coli is the most commonly used bacterium for production of heterologous proteins, the expression of recombinant antigens in bacterial systems other than E. coli may sometimes be advantageous. Salmonella typhimurium, V. cholerae and Bacillus brevis are some examples of other bacteria that have been used for expression of antigens for vaccine production purposes.

Known expression systems using Vibrio cholerae host cells for the production of heterologous proteins include but are not limited to the CTB expression system disclosed in Sanchez and Holmgren, Proc. Natl. Acad. Sci. USA 1989: 86: 481-5. Details of this expression system are also disclosed in U.S. Pat. Nos. 5,268,276, 5,834,246, 6,043,057 and EP Patent No 0368819. In this expression system, the CTB subunit is obtained by expressing the gene encoding cholera toxin B subunit in a V. cholerae host cell in the absence of a V. cholerae gene encoding the A subunit of cholera toxin (CTA).

Lebens et al (1993 Biotech 11; 1574-8) described a modification of the method of Sanchez and Holmgren (1989 ibid) for preparing CTB. In this regard, recombinant CTB was produced by a mutant strain of V. cholerae 01, deleted of its CT genes and transfected with a multicopy plasmid encoding CTB. The CTB used was purified from the culture medium by a combination of salt precipitation and chromatographic methods, as described.

The use of bacterial host cell, such as V. cholerae host cells for expression of recombinant proteins as demonstrated by Sanchez and Holmgren (1989) (ibid) and Lebens et al (19930 (ibid) has been shown to be advantageous over other prokaryotic expression systems in common use in that specific recombinant products may be produced in large quantities and secreted into the culture medium, thereby facilitating downstream purification procedures. This efficient secretion of CTB from V. cholerae host cells is different from the secretory process from E. coli cells where the expressed product often assembles in the periplasmic space (Neill et al. 1983 Science. 221: 289-290). However, recently, a protein secretory pathway for the secretion of heat-labile enterotoxin (LT) by an enterotoxigenic strain of E. coli has been identified (Tauschek et al (2002) PNAS 99: 7066-7071) which envisage the efficient secretion of a recombinant protein from an E. coli host cell.

Whilst the expression system disclosed in Sanchez and Holmgren 1989 (ibid) and Lebens et al (ibid) appear to produce CTB at acceptable levels, these expression systems suffer from the disadvantage that an antibiotic, such as ampicillin, is required in the culture medium to maintain optimum production by selecting for and maintaining plasmids comprising a gene of interest. In the absence of ampicillin, the plasmid containing the gene encoding the CTB subunit protein would not be stably maintained and the yield of the CTB would decrease. In addition, a further downstream processing step is required to effectively remove all the antibiotic residues from the purified product.

The use of antibiotics in the production of recombinant proteins is undesirable for a number of reasons. Apart from the obvious increase in costs arising from the need to add antibiotics as a supplement to the growth medium, the use of antibiotics is considered a problem in the production of any recombinant protein intended for human or veterinary use. This is primarily for three reasons. Firstly, residual antibiotics may cause severe allergic reactions in sensitive individuals. Secondly, there is the possibility of selection for antibiotic resistant bacteria in the natural bacterial flora of those using the product. Finally, DNA encoding the antibiotic resistance may also be transferred to sensitive bacteria in individuals using the product, thereby also spreading undesired antibiotic resistance in a cohort.

As the large scale production of recombinant proteins, such as CTB, which are free of antibiotic residues, is commercially important in the pharmaceutical industry, there is a need to provide pharmaceutically acceptable CTB at as high yields as possible.

### SHORT DESCRIPTION OF THE INVENTION

This present invention teaches how to improve CTB yields using a CTB production system comprising a Vibrio cholerae host cell lacking the functionality of a thyA gene which is used in conjunction with a novel expression vector to produce unexpected high yields of CTB relative to the yields obtained with known bacterial host cell production systems.

A plasmid expression vector was constructed in which a gene encoding a thymidylate synthase enzyme (thyA) gene was used as a means of selection and maintenance of a plasmid comprising a CTB gene. The plasmid is of reduced size relative to known expression plasmids for producing CTB because substantially all of the non-coding V. cholerae DNA downstream of the CTB gene is removed.

The unexpected high yield of CTB obtained using this expression system demonstrated both the efficiency of expression of a heterologous gene in a V. cholerae host cell and the stability of the plasmids maintained by complementation of a thyA deletion in the V. cholerae host cell strain. By way of example, even after repeated passages through liquid culture equivalent to 100 generations, all the cells retained the plasmid and the ability to express the recombinant protein.

The expression system as reported here is advantageous because it facilitates the production of CTB for the following uses which include, but are not limited to:

a protective immunogen in oral vaccination against cholera and LT-caused E. coli diarrhoea;

An immunomodulator or a tolerogenic inducing agent or an immune-deviating agent for down-regulating, modulating, de-sensitising or re-directing the immune response;

An adjuvant for altering, enhancing, directing, re-directing, potentiating or initiating an antigen-specific or non-specific immune response;

A carrier to stimulating an immune response to one or more unrelated antigens; and

A diagnostic agent for producing antibodies (such as monoclonal or polyclonal antibodies) for use in diagnostic or immunodiagnostic tests.

It is a particular advantage from the point of purification and standardisation of CTB as a vaccine component that relatively high yields of CTB can be achieved using stable bacterial host cell strains that lack the functionality of a thyA gene.

The present invention provides, in particular, an expression system for producing a B subunit of a cholera toxin (CTB) wherein the expression system comprises:
(a) a Vibrio cholerae host cell lacking the functionality of a thyA gene; and
(b) an expression vector less than 5 kb in size comprising a functional thyA gene and a CTB gene which is substantially free of the flanking sequences immediately contiguous by the 5′ and 3′ end of the CTB gene in the naturally occurring genome of the host cell from which the CTB gene is derived.

In an embodiment of the expression system according to the invention, the host cell lacks the functionality of a CTA gene. In another embodiment the expression vector is about 3 kb in size. In a further embodiment the expression vector comprises an E. coli thyA gene. In yet another embodiment the expression vector has the nucleotide sequence presented in SEQ ID NO: 1. In still another embodiment the expression vector further comprises at least one further nucleotide sequence encoding a heterologous protein, such as a non-toxic component or form of the heat labile E. coli enterotoxin LT, preferably the non-toxic component of LT is the B subunit of a (LTB) or a fragment thereof.

The invention is also directed to a method of producing CTB wherein the method comprises:

transforming a Vibrio cholerae host cell lacking the functionality of a thyA gene with an expression vector less than 5 kb in size comprising a functional thyA gene and a CTB gene which is substantially free of the flanking sequences immediately contiguous by the 5′ and 3′ end of the CTB gene in the naturally occurring genome of the host cell from which the CTB gene is derived, and

culturing the transformed V. cholerae host cell under conditions which permit production of the CTB, optionally followed by isolating and/or purifying the CTB from the host cell.

The expression vector used in the expression system and the method of the invention is composed of a novel nucleic acid construct.

Thus, the invention is further directed to an isolated nucleic acid construct which comprises a thyA gene and a CTB gene which is substantially free of the flanking sequences immediately contiguous by the 5′ and 3′ end of the CTB gene in the naturally occurring genome of the host cell from which the CTB gene is derived, and which nucleic acid construct is less than 5 kb in size.

In an embodiment of the nucleic acid construct according to the invention, the nucleic acid construct is about 3 kb in size. In another embodiment, the nucleic acid construct is a plasmid, such as pMT-ctxBthyA-2 characterised by a restriction endonuclease map as shown in FIG. 13. In yet another embodiment the plasmid has the nucleotide sequence SEQ ID NO: 1.

Thus, the present invention provides a novel and improved stable expression system comprising a combination of (i) a stable V. cholerae host cell strain lacking the functionality of a thyA gene; and (ii) a stable expression vector comprising a functional thyA gene and a CTB gene which is substantially free of the flanking sequences immediately contiguous by the 5′ and 3′ end of the CTB gene in the naturally occurring genome of the host cell from which the CTB gene is derived.

The stable expression system is advantageous because it;

(i) ensures the stable maintenance of the CTB encoding plasmid (by ensuring, for example, a 100% plasmid retention in the large production fermentor) which is advantageous because it ensures a consistent and reliable production of CTB; and

(ii) improves on CTB quality by eliminating the heterogeneity found in the N-terminus of CTB which ensures consistent production of the same CTB end product.

The invention also provides an isolated stable expression vector for producing CTB which is an improvement over the known expression vectors for producing CTB while still comprising a functional thyA gene. The expression plasmid is of reduced size because it eliminates substantially all of the V. cholerae DNA downstream of the CTB gene. Without wishing to be bound by theory, it is believed that removing substantially all of non-coding V. cholerae DNA downstream of the ctxB gene resulting in the reduced size of the expression vector contributes to the improved stability and the improved yield of the CTB product. By way of example of the improved plasmid stability, when V. cholerae host cells are used in the expression system, almost all the V. cholerae cells retained (i) the plasmid comprising the CTB gene and (ii) the ability to express the recombinant CTB protein even after repeated passage through liquid culture equivalent to 100 generations.

The presence of a functional thyA gene in the expression vector is advantageous because:

It complements the thyA deficiency in the V. cholerae host strain;

It enables the strain to grow in the absence of thymine in the growth medium; and

It ensures the genetic stability of the V. cholerae host strain when grown in a medium devoid of extraneous thymine since loss of the plasmid leads to death of the host strain.

For some embodiments, the nucleotide sequence encoding the functional thymidylate synthase (thyA) enzyme is an E. coli nucleotide sequence or derivable from E. coli. The use of plasmid comprising a nucleotide sequence encoding a thyA enzyme derivable from E. coli is advantageous because the V. cholerae thyA gene has only about 30% homology with the corresponding thyA sequence from E. coli so the risk of a recombination event is reduced.

The method for producing a cholera toxin B (CTB) subunit protein which comprises introducing the defined stable expression vector into a V. cholerae host cell lacking the functionality of a thyA gene, and cultivating the host cell under conditions whereby CTB is produced gives the following advantages:
(i) improved yield of CTB such that the yield of CTB from the expression system is increased 4-5 fold relative to known CTB expression systems (for example, levels of CTB produced using the known CTB expression systems as described in Sanchez and Holmgren (1989) (ibid);
(ii) simplification of the production process for CTB because the downstream step of removing antibiotic residues from CTB can be eliminated. The simplification of the production process results in a cheaper product because there is a reduction in costs in the large scale production of the protein and of the elimination of the need for “down stream processing step” to remove any antibiotic residues from the expressed CTB product.

Other aspects of the present invention will be apparent to those of ordinary skill in the art from the accompanying claims and the following description and drawings.

### DETAILED DESCRIPTION

Before describing the present invention in detail, it is to be understood that this invention is not limited to particularly exemplified molecules or process parameters as such may, of course, vary. It is also to be understood that the terminology used herein is for the purpose of describing particular embodiments of the invention only, and is not intended to be limiting. In addition, the practice of the present invention will employ, unless otherwise indicated, conventional methods of virology, microbiology, molecular biology, recombinant DNA techniques and immunology all of which are within the ordinary skill of the art. Such techniques are explained fully in the literature. See, e.g., Sambrook, et al., Molecular Cloning: A Laboratory Manual (2nd Edition, 1989); DNA Cloning: A Practical Approach, vol. I & II (D. Glover, ed.); Oligonucleotide Synthesis (N. Gait, ed., 1984); A Practical Guide to Molecular Cloning (1984); and Fundamental Virology, 2nd Edition, vol. I & II (B. N. Fields and D. M. Knipe, eds.).

All publications, patents and patent applications cited herein, whether supra or infra, are hereby incorporated by reference in their entirety. It must be noted that, as used in this specification and the appended claims, the singular forms “a”, “an” and “the” include plural referents unless the content clearly dictates otherwise.

For the avoidance of doubt, the term “comprising” encompasses “including” as well as “consisting”. By way of example, a composition “comprising” X may consist exclusively of X or may include something additional to X such as X and Y.

All scientific and technical terms used in this application have meanings commonly used in the art unless otherwise specified. The standard nomenclature as used in, for example, E-L Winnacker, From Genes to Clones, VCH Publishers, New York (1987) is adhered to for defining DNA restriction endonucleases, restriction sites and restriction sequences. Oligodeoxynucleotides and amino acids are referred to with the conventional one-letter and three-letter abbreviation codes. The one-letter amino acid symbols recommended by the IUPAC-IUB Biochemical Nomenclature Commission are provided at the beginning of the Example Section.

As used in this application, the following words or phrases have the meanings specified.

Expression System

The present invention relates to a stable expression system comprising a host cell and expression vector combination to produce CTB.

The term “expression system” refers to a combination of a host cell and a compatible expression vector which are maintained under suitable conditions, such as, for example, the expression of a protein coded for by foreign DNA carried by the vector and introduced into the host cell. In the case of the expression system as described herein, a thyA gene which is essential for bacterial survival is rendered non-functional on the bacterial host cell chromosome. A functional thyA gene is provided on a complementing plasmid. The thyA gene acts as a selection marker since loss of the plasmid will therefore mean the bacterial host cell is unable to survive. The selection of a thyA gene as the non-antibiotic selection marker provides particular advantages as outlined above.

The terms “express” and “expression” includes allowing or causing the information in a gene or DNA sequence to become manifest, for example by producing RNA (such as rRNA or mRNA) or by producing a protein by activating the cellular functions involved in transcription and translation of a corresponding gene or DNA sequence. A DNA sequence is expressed by a cell to form an “expression product” such as an RNA (such as, for example, a mRNA or a rRNA) or a protein. The expression product itself, such as, for example, the resulting RNA or protein, may also said to be “expressed” by the cell.

Host Cell

As used herein, the term “host cell” refers to any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells can further be used for screening or other assays. The term “host cells” may denote, for example, bacterial cells, that can be, or have been, used as recipients for recombinant vector or other transfer DNA, and include the progeny of the original cell which has been transformed. It is understood that the progeny of a single parental cell may not necessarily be completely identical in morphology or in genomic or total DNA complement as the original parent, due to natural, accidental, or deliberate mutation. By way of example, the CTB of the present invention may be expressed in V. cholerae host cells.

In one embodiment, the present invention relates to a CTB production system comprising a V. cholerae bacterial host cell lacking the functionality of a thyA gene which is used in conjunction with a novel expression vector to produce unexpected high yields of CTB relative to the yields obtained with known bacterial host cell production systems.

Vibrio Cholerae Host Cells

It is well known in the art that V. cholerae of serogroup O1 and 0139 may induce severe diarrhoeal disease when multiplying in the gut of infected individuals by releasing cholera toxin (CT) which induces active electrolyte and water secretion from the intestinal epithelium. By analogous mechanisms several other bacteria, for instance enterotoxigenic E. coli bacteria (ETEC), may also cause diarrhoea by releasing other enterotoxins that may be related or unrelated to CT.

CT is the prototype bacterial enterotoxin. It is a protein built from two types of subunits: a single A subunit of molecular weight 28,000 and five B subunits, each with a molecular weight of 11,600. The B subunits are aggregated in a ring by tight noncovalent bonds; the A subunit is linked to and probably partially inserted in the B pentamer ring through weaker noncovalent interactions. The two types of subunits have different roles in the intoxication process: the B subunits are responsible for cell binding and the A subunit for the direct toxic activity.

The molecular aspects of toxin binding to intestinal and other mammalian cells and of the subsequent events leading to activation of adenylate cyclase through the intracellular action of the A subunit (and its A1 fragment) have been clarified in considerable detail (see J Holmgren, Nature 292:413-417, 1981). More recently information has also become available on the genetics and biochemistry of cholera toxin synthesis, assembly and secretion by V. cholerae bacteria.

CT is encoded by chromosomal structural genes for the A and B subunits, respectively. These genes have been cloned from several strains, and their nucleotide sequences have been determined (see for example, Heidelberg et al (2000) Nature 406: 477-483). The genes for the A and B subunits of CT are arranged in a single transcriptional unit with the A cistron (ctxA) preceeding the B cistron (ctxB). Studies on the organization of CT genes in V. cholerae strains of classical and EI Tor biotypes have suggested that there are two copies of CT genes in classical biotype strains while there is only one copy in most EI Tor strains (J J Mekalanos et al, Nature 306:551-557, 1983). The synthesis of CT is positively regulated by a gene, toxR that increases ctx expression manifold (V L Miller and J J Mekalanos, Proc Natl Acad Sci USA, 81:3471-3475, 1984). ToxR acts at the transcriptional level, and is present in strains of both classical and EI Tor biotypes. ToxR probably increases ctx transcription by encoding a regulatory protein that interacts positively with the ctx promoter region.

A V. cholerae host cell strain lacking the functionality of a thyA gene can be prepared by methods of the invention or by methods known to those skilled in the art (Sambrook, J. E. F. Fritsch, and T. Maniatis, Molecular cloning: a laboratory manual. 2nd ed. 1989: Cold Spring Harbor Laboratory Press, Cold Spring Harbor, N.Y.). Appropriate known methods for preparing the V. cholerae strain lacking the functionality of the thyA gene include the methods outlined in WO 99/61634.

In the context of the present invention, a thyA gene lacks functionality if, for example, the gene has been removed—such as by deletion—or if the gene has been genetically disabled by, for example, inactivation or site directed mutagenesis of the thyA gene so that there is no expression of the thyA enzyme. The lack of functionality of a thyA gene may be determined, for example, by transforming a thyA negative vector with a thyA positive gene and selecting for absence of growth in the absence of thymine.

thyA Selectable Marker System

The complementation of a chromosomal lesion on a bacterial host cell strain has been used as means of plasmid maintenance. Thus, the non-functional thyA gene on the V. cholerae chromosome is complemented by the presence of a functional thyA gene provided on a complementary plasmid which acts as a selectable marker and which eliminates the need to an antibiotic resistance selection marker. The thyA gene also acts as a selectable marker in the sense that loss of the plasmid means that the V. cholerae bacterium is unable to survive.

The thymidylate synthetase (thyA) enzyme encoded by the thyA gene of V. cholerae, E. coli and other bacteria catalyses the methylation of deoxyuridylate (dUMP) to deoxythymidylate (dTMP) and is an essential enzyme in the biosynthesis of deoxyribothymidine triphosphate (dTTP) for incorporation into DNA. In the absence of this enzyme the bacteria become dependent upon an external source of thymine which is incorporated into dTTP by a salvage pathway encoded by the deo genes (Milton et al 1992 J Bacteriol 174: 7235-7244).

It is known that the thyA gene is a conserved gene and can be found in bacteriophages, prokaryotes and eukaryotes. Because the thyA enzyme is conserved, the thyA gene, from, for example, E. coli, is able to complement the mutant thyA genes located in the chromosome of related bacterial species as discussed below. The functional thyA gene in the plasmid may be from sources other than E. coli. In one embodiment, wherein the host cell is a V. cholerae host cell, the thyA gene has a low homology with the V. cholerae thyA gene.

Previous work in the field has demonstrated that recombinant plasmids can be maintained in V. cholerae in the absence of antibiotic selection by complementation of a thyA mutation with the thyA gene from E. coli. The principle was demonstrated initially using plasmids carrying the E. coli thyA gene and spontaneous thyA mutants of V. cholerae isolated on the basis of resistance to trimethoprim (Morona et al 1991 Gene 107: 139-144).

Further work by Carlin and co-workers resulted in the cloning and characterisation of the thyA locus from V. cholerae and the generation of stable defined recipient V. cholerae strains. The sequence of the V. cholerae thyA gene as determined by Carlin and co-workers is published in EMBL (Genebank Accession No AJ006514). WO 99/61634 teaches that defined thyA mutants of V. cholerae may be used as suitable production strains for recombinant proteins encoded on plasmids maintained by thyA complementation.

The use of a thyA gene on the complementing plasmid, which has low homology with the V. cholerae thyA gene is advantageous because the risk of “cross-over” with the V. cholerae chromosome is reduced.

Preferably the thyA gene on the complementing plasmid is an E. coli thyA gene which has a low homology with V. cholerae thyA gene. By way of explanation, the published sequence for the E. coli thyA gene can be found at Genebank Accession No J01709. A comparison of the sequence of the V. cholerae thyA gene (protein of 283 amino acids) as determined by Carlin et al (see Genebank Accession No AJ006514) and the E. coli thyA gene (see Genebank Accession No J01709) showed only 32% amino acid identity and reflects only about 54% homology in a 454 bp overlap at the DNA level (see FIG. 7 of WO 99/61634). In this regard, homology searches of the EMBL DNA and Swiss-Prot protein data libraries were done by the FASTA software in the GCG program package (Wisconsin Package Version 9.0, Genetics Computer Group (GCG), Madison, Wis.)

The expression of CTB as described herein may be driven by a variety of promoters. Preferably the promoter is a heterologous promoter. As used herein, the term “heterologous” refers to two biological components that are not found together in nature. The components may be regulatory regions, such as promoters. As used herein, the term “heterologous promoter” refers to a promoter which is unrelated to the gene with which is it operably linked. Preferably the promoter is a heterologous prokaryotic promoter. In particular, preferably the promoter is a promoter suitable for the host cell in which it will be used. More preferably, expression of the CTB gene In the expression system as described herein is driven by the tacP Promoter or T7 RNA polymerase dependent promoter. In one embodiment, CTB may be expressed in an inducible (such that a stimulus is required to initiate expression) or a constitutive manner (such that it is continually produced) under the control of a heterologous promoter, such as the tacP promoter. In the case of inducible expression, the production of rCTB can be initiated when required by, for example, addition of an inducer substance to the culture medium, for example dexamethasone or Isopropylthiogalactoside (IPTG) which is an artificial inducer of the Lac operon.

Any suitable transcriptional termination sequence may be used, preferably a strong transcriptional termination sequence which allows minimal or no transcription. In a preferred embodiment of the invention, TrpA terminators are located downstream of the CTB gene effectively terminating mRNA transcription. In one embodiment described in the Examples, the nucleotide sequences of the transcription terminator sequences are shown in FIG. 14 (from about nucleotide 2732 to about nucleotide 2759).

Fusion proteins provide an alternative to direct expression. Usually, a DNA sequence encoding the N-terminal portion of an endogenous bacterial protein, or other stable protein, is fused to the 5′ end of heterologous coding sequences. Upon expression, this construct will provide a fusion of the two amino acid sequences. For example foreign proteins can also be secreted from the cell by creating chimeric DNA molecules that encode a fusion protein comprised of a signal/leader peptide sequence fragment that provides for secretion of the foreign protein in bacteria [see for example U.S. Pat. No. 4,336,336]. The signal/leader sequence fragment usually encodes a signal peptide comprised of hydrophobic amino acids which direct the secretion of the protein from the cell. The protein is either secreted into the growth media (such as, for example, for gram-positive bacteria) or into the periplasmic space, located between the inner and outer membrane of the cell (such as, for example, for gram-negative bacteria). Preferably there are processing sites, which can be cleaved either in vivo or in vitro encoded between the signal peptide fragment and the foreign gene.

DNA encoding suitable signal sequences can be derived from genes for secreted bacterial proteins, such as the E. coli outer membrane protein gene (ompA) [Masui et al. (1983), in: Experimental Manipulation of Gene Expression; Ghrayeb et al. (1984) EMBO J. 3:2437] and the E. coli alkaline phosphatase signal sequence (phoA) [Oka et al. (1985) Proc. Natl. Acad. Sci. 82:7212]. As an additional example, the signal sequence of the alpha-amylase gene from various Bacillus strains can be used to secrete heterologous proteins from B. subtilis [Palva et al. (1982) Proc. Natl. Acad. Sci. USA 79:5582; EPO Publ. No. 244 042].

As used herein, the term “leader sequence” or “signal sequence” relates to any nucleotide encoding sequence or encoded peptide sequence on a protein molecule which facilitates the translocation or exportation of a protein, such as the translocation or exportation of an expressed CTB protein across the cellular membrane and cell wall, if present, or at least through the cellular membrane into the periplasmic space of a cell having a cell wall. As used herein, the term “leader sequence” or “signal sequence” refers to a DNA sequence that encodes a polypeptide (a “secretory peptide”) that, as a component of a larger polypeptide or propeptide sequence, which directs the larger polypeptide through a secretory pathway of a cell (such as from the endoplasmatic reticulum to the Golgi apparatus and further to a secretory vesicle) in which it is synthesized. The larger polypeptide is commonly cleaved to remove the secretory peptide during transmit through the secretory pathway. The secretory signal sequence may encode any signal peptide which ensures efficient direction of the expressed polypeptide into the secretory pathway of the cell. The signal peptide may be naturally occurring signal peptide, or a functional part thereof, or it may be a synthetic peptide.

In one embodiment, the leader sequence is from an enterotoxin, such as an E. coli heat-labile enterotoxin (LT) leader sequence. Examples of LT leader sequences are provided in the sequences listed in Table 1 under their listed G1 Accession numbers. In one preferred embodiment, the leader sequence is E. coli heat-labile enterotoxin (LTB) leader sequence). The LTB signal sequence for producing CTB of the present invention is presented in Table 2 as MNKVKFYVLFTA LLSS LCAHG (SEQ ID NO: 2). Other examples of leader sequences include but are not limited to leader sequences presented in Table 2 and as part of the sequences presented in FIG. 14.

In the described examples, the CTB gene is fused to the LTB signal peptide from the heat-labile enterotoxin of E. coli in such a way that the naturally occurring Sacl site can be used.

DNA Construct

Usually, the above described components, comprising a promoter, signal sequence (if desired), coding sequence of interest, and transcription termination sequence, are put together into expression constructs. A segment or sequence of DNA having inserted or added DNA, such as an expression vector, may also be called a “DNA construct” or a “nucleic acid construct”. Enhancers, introns with functional splice donor and acceptor sites, and leader sequences may also be included in an expression construct, if desired. Expression constructs are often maintained in a replicon, such as an extrachromosomal element (e.g., plasmids) capable of stable maintenance in a host, such as bacteria. The replicon will have a replication system, thus allowing it to be maintained in a procaryotic host either for expression or for cloning and amplification. In addition, a replicon may be either a high or low copy number plasmid. A high copy number plasmid will generally have a copy number ranging from about 5 to about 200, and usually about 10 to about 150. A host containing a high copy number plasmid will preferably contain at least about 10, and more preferably at least about 20 plasmids. Either a high or low copy number vector may be selected, depending upon the effect of the vector and the foreign expressed protein on the host cell. Alternatively, some of the above described components can be put together in transformation vectors. Transformation vectors are usually comprised of a selectable marker that is either maintained in a replicon or developed into an integrating vector, as described above.

Replicon

As used herein, a “replicon” is any genetic element, e.g., a plasmid, a chromosome, a virus, a cosmid, and the like that behaves as an autonomous unit of polynucleotide replication within a cell. The replicon is capable of replication under its own control and may include selectable markers.

Vector

As used herein, a “vector” is a replicon in which another polynucleotide segment is attached, so as to bring about the replication and/or expression of the attached segment. The term “vector” includes expression vectors and/or transformation vectors. The term “expression vector” means a construct capable of in vivo or in vitro/ex vivo expression. The term “transformation vector” means a construct capable of being transferred from one species to another. Examples of vectors include but are not limited to plasmids, chromosomes, artificial chromosomes or viruses.

Plasmids

A common type of vector is a “plasmid”, which generally is a self-contained molecule of double-stranded DNA, usually of bacterial origin, that can readily accept additional (such as heterologous) DNA and which can readily introduced into a suitable host cell. A large number of vectors, including plasmid and fungal vectors, have been described for replication and/or expression in a variety of eukaryotic and prokaryotic hosts. The plasmid employed in the invention may be a plasmid known in the art such as but not limited to plasmids such as pBR322, pACYC177 or pUC plasmid derivatives or the pBLUESCRIPT vector (Stratagene, La Jolla, Calif.).

Plasmids such as pJS162 (as described in Sanchez and Holmgren (1989) (ibid) and (pML358) (as described in Lebens et a/1993 ibid) have been used to produce CTB in a V. cholerae host cell expression systems. The expression vector of the present invention is different from the expression plasmids of Sanchez-Holmgren (pJS162) and Lebens (pML358) in that:
(i) the plasmid is of a smaller size because substantially all of the non-coding V. cholerae DNA downstream of the CTB gene has been removed; and
(ii) the plasmid has a functional thyA gene.

In this regard, Table 3 provides a comparative analysis of the expression vector as described herein with the relevant expression vectors known in the art. In one embodiment, the stable expression vector as described herein is preferably less than 5 kb in size. In a more preferred embodiment, the stable expression vector is from about 2.5 kb to 4 kb in size. In an even more preferred embodiment, the stable expression vector is about 3 kb in size.

The term “about” or “approximately” means within an acceptable error range for the particular value as determined by one of ordinary skill in the art, which will depend in part on how the value is measured or determined within the limitations of the measurement system. For example, “about” can mean within 1 or more than 1 standard deviations, as per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The smaller plasmid size is advantageous because it allows easier in vitro manipulation and construction of derivatives because smaller DNA molecules ligate together and transform into prokaryotic hosts, such as V. cholerae, more efficiently, improving the chances of obtaining derivatives of the correct construction. The smaller size also allows greater efficiency when introducing the constructs into recipient bacteria by, for example, transformation and also to increase the stability of the plasmid.

Expression Vector

As used herein, the term “expression vector” means the vehicle by which a nucleotide sequence (such as, a heterologous nucleotide sequence) can be introduced into a host cell so as to transform the host and promote expression (such as, for example, transcription and translation) of the introduced sequence.

Isolated Expression Vector

As used herein, the term “expression vector” includes an isolated expression vector as well as an expression vector which is part of a host cell/expression vector combination. The terms “isolated” and “purified” refer to molecules, either nucleic or amino acid sequences or nucleic acid constructs that are removed from their natural environment and/or isolated or separated from at least one other component with which they are naturally associated. By way of example, an expression vector may be regarded as “isolated” if it has been prepared under conditions that reduce or eliminate the presence of unrelated materials, such as, for example, contaminants, including native materials from which the material is obtained. By way of further example, a purified protein is regarded as isolated if it is substantially free of other proteins or nucleic acids with which it is associated in a cell. Likewise, a purified nucleic acid molecule is isolated if it is substantially free of proteins or other unrelated nucleic acid molecules with which it can be found within a cell. A protein may be mixed with carriers or diluents which will not interfere with the intended purpose of the substance and still be regarded as substantially isolated.

Heterologous Nucleotide Sequence

Generally, a heterologous nucleotide sequence is inserted at one or more restriction sites of the vector DNA, and then is carried by the vector into a host cell along with the transmissible vector DNA. As used herein, the term “heterologous nucleotide sequence” refers to a nucleotide sequence which is not naturally located in a cell or in a chromosomal site of a cell or which is not naturally expressed by a cell. As used herein, x is “heterologous” with respect to y if x is not naturally associated with y in the identical manner; i.e., x is not associated with y in nature or x is not associated with y in the same manner as is found in nature. The term “heterologous nucleotide sequence” is used interchangeably with the terms “foreign” nucleotide sequence or “guest” nucleotide sequence or “extracellular” nucleotide sequence or “extrinsic” or “exogenous” nucleotide sequence throughout the text. The heterologous nucleotide sequence may also be a coding sequence.

As used herein, the terms “gene”, “coding sequence” or a nucleotide sequence “encoding” an expression product, such as a RNA, polypeptide, protein or enzyme, refers to a nucleotide sequence which when expressed, results in the production of that RNA, polypeptide, protein or enzyme. That is, the nucleotide sequence “encodes” that RNA or it encodes the amino acid sequence for that polypeptide, protein or enzyme. The term “nucleotide sequence” is synonymous with the term “polynucleotide”. A gene sequence or nucleotide sequence is “under the control of” or is “operably linked with” transcriptional and translational control sequences in a cell when RNA polymerase transcribes the coding sequence into RNA, which is then trans-RNA spliced (if it contains introns) and, if the sequence encodes a protein, is translated into that protein. The nucleotide sequence may be DNA or RNA of genomic or synthetic or of recombinant origin. The nucleotide sequence may be double-stranded or single-stranded whether representing the sense or antisense strand or combinations thereof.

Coding Sequence

As used herein, a “coding sequence” is a polynucleotide sequence which is translated into a polypeptide, usually via mRNA, when placed under the control of appropriate regulatory sequences. The boundaries of the coding sequence are determined by a translation start codon at the 5′-terminus and a translation stop codon at the 3′-terminus. A coding sequence can include, but is not limited to, cDNA, and recombinant polynucleotide sequences. An “open reading frame” (ORF) is a region of a polynucleotide sequence which encodes a polypeptide; this region may represent a portion of a coding sequence or a total coding sequence.

Operably Linked

A control sequence may be operably linked to a coding sequence. As used herein, the term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

Cholera Toxin (CT) and B Subunit Thereof (CTB)

As used herein, the term “CT” refers to the cholera toxin and “CTB” refers to the B subunit of the cholera toxin. In other texts, these may sometimes be identified as “CT” or “Ct” and “CtxB” or “CtB” respectively. The CTB produced by the expression system of the present invention may also be referred to as recombinant CTB (rCTB).

The term “CTB” also includes recombinant CTB DNA sequences which are part of a hybrid CTB gene or derivative thereof encoding additional sequences. A CTB derivative could be a fusion protein such as a CTB gene fusion protein or a CTB coupled with other elements.

Heat-Labile Enterotoxin (LT) and B Subunit Thereof (LTB)

As used herein, the term “LT” herein refers to the E. coli heat labile enterotoxin, and “LTB” is the B subunit of LT. In other texts, these may sometimes be identified as “Etx” or “Et” and “EtB” or “EtxB” respectively. The heat-labile toxin (LT) of enterotoxigenic E. coli (ETEC) is structurally, functionally and immunologically similar to CTB. The two toxins cross-react immunologically.

CTB Gene

The CTB gene or nucleotide sequence encoding CTB is substantially free from the flanking sequences immediately contiguous by the 5′ and 3′ end of the CTB encoding sequence in the naturally occurring genome of the micro-organisms from which the CTB encoding DNA is derived. In other words, the CTB gene is substantially free of the 5′ and 3′ flanking sequences homologous to its host cell genome. For some applications, the CTB gene or the nucleotide sequence encoding the CTB protein may be the same as the naturally occurring or native form or wild type form of CTB.

“Native CTB”

As used herein the term “native CTB” refers to a CTB molecule with properties, such as activity (such as, for example, GM-1 binding activity) and/or Immunogenic and/or immunomodulatory properties which are substantially the same as the naturally occurring form or wild type form of the CTB molecule which is capable of binding to GM1 and/or which have the immunogenic or immunomodulatory capability of the CTB molecule. The terms “native”, “naturally occurring”, “wild-type” form of CTB are used inter-changeably throughout the text.

In one embodiment (as described in the Examples below), the substantially pure CTB gene is presented as the nucleotide sequence from about nucleotides 2402 to about nucleotides 2710 in FIG. 14.

For some applications, the CTB gene or the nucleotide sequence encoding the CTB protein may be a variants, homologues, derivatives or fragments thereof of the naturally occurring or native form of the CTB.

As used herein, the term “variants, homologues, derivatives and fragments thereof” of a native CTB molecule include CTB molecules which may be structurally different from the native CTB molecule (such as, for example, in terms of nucleotide sequence) but which behave functionally like the native CTB molecule particularly in terms of its binding properties, such as binding to GM1 ganglioside and/or its immunological properties such as reacting with antiserum to CTB as detected by an ELISA or GM1-ELISA test. These variants, homologues, derivatives and fragments thereof of a native CTB molecule include but are not limited to the B subunit of heat-labile enterotoxin from E. coli B (LTB) and to any or all mutated, extended, truncated or otherwise modified forms of B subunits or any other protein that would react with GM1 or with said types of antisera as well as any nucleic acid preparation that would encode for a protein that would meet these criteria but which do not have any ADP-ribosylating activity.

In another embodiment, the CTB gene is presented as a variant, homologue, derivative or fragment of the sequence presented from about nucleotide 2402 to about nucleotide 2710 in FIG. 14.

“Mature CTB”

As used herein, the term “mature CTB” refers to the expressed CTB subunit protein which is devoid of a signal sequence.

As used herein, the term “amino acid sequence” refers to peptide, polypeptide sequences, protein sequences or portions thereof.

As used herein, the term “protein” is synonymous with the term “amino acid sequence” and/or the term “polypeptide”. In some instances, the term “amino acid sequence” is synonymous with the term “peptide”. In some instances, the term “amino acid sequence” is synonymous with the term “protein”.

As used herein, the term “polypeptide” refers to a polymer of amino acids and does not refer to a specific length of the product. Thus, peptides, oligopeptides, and proteins are included within the definition of polypeptide. This term also does not refer to or exclude post expression modifications of the polypeptide, for example, glycosylations, acetylations, phosphorylations and the like. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring.

A polypeptide or amino acid sequence “derived from” a designated nucleic acid sequence refers to a polypeptide having an amino acid sequence identical to that of a polypeptide encoded in the sequence, or a portion thereof wherein the portion consists of at least 3-5 amino acids, and more preferably at least 8-10 amino acids, and even more preferably at least 11-15 amino acids, or which is immunologically identifiable with a polypeptide encoded in the sequence. This terminology also includes a polypeptide expressed from a designated nucleic acid sequence.

N-Terminal Mutation

Threonine (T) is the first amino acid normally found in mature CTB from the native or naturally occurring form of the CTB molecule. Thus, generally, the N-terminal sequence for mature CTB molecule is Thr-Pro-Gln-Asn-lle-Thr (TPQNIT) (SEQ ID NO: 3). Examples of mature CTB molecules with a TPQNIT N-terminal sequence include but are not limited to the CTB amino acid sequence from V. cholerae strain 0395, classical Ogawa, which is shown in U.S. Pat. Nos. 5,268,276, 5,823,4246 and 6043057, EP Patent No 0368819 and Figure 2 of Sanchez and Holmgren (1989) (ibid). The described embodiments provide an example of a CTB sequence with a TPQNIT N-terminal sequence which is produced using a V. cholerae host cell expression system.

In one embodiment, variants of the CTB sequence may be used which advantageously have the APQNIT (Ala-Pro-Gln-Asn-Ile-Thr) (SEQ ID NO: 4) N-terminal sequence. By way of example, the CTB sequence SEQ ID NO: 1, also presented in FIG. 14, is the same as the CTB native sequence from V. cholerae strain 0395, classical Ogawa, apart from the single mutation at the amino terminal end of the protein sequence where an alanine (Ala) residue is introduced at the first position of the CTB amino acid sequence instead of a Threonine (Thr=T) Introduction of this particular amino acid (Ala) is advantageous because it creates a defined signal sequence cleavage site, as opposed to the threonine (Thr) residue at the amino terminus of the wild type or native form of CTB. This cleavage site can be important in post-translational modifications. This N-terminal mutation is advantageous because it improves on CTB quality by eliminating the heterogeneity found in the N-terminus of CTB produced using known CTB expression systems (such as the CTB expression system described in Sanchez and Holmgren 1989 (ibid) and so ensures consistent production of the same CTB end product. In this respect, the junction of the eltBlctxB gene has been modified so that only a single N-terminal is obtained in the resulting CTB protein, in comparison up to about two different N-termini which are obtained with the native CTB molecule (see U.S. Pat. Nos. 5,268,276, 5,823,4246 and 6,043,057, EP Patent No 0368819 and Sanchez and Holmgren (1989) (ibid).

Variant

As used herein, the term “variant” may also be used to indicate a modified or altered gene, DNA sequence, RNA, enzyme, cell and the like which differs from the native type sequence. A variant may be found within the same bacterial strain or may be found within a different strains. Preferably the variant has at least 90% sequence identity with the native or naturally occurring form of the CTB sequence. Preferably the variant has 20 mutations or less over the whole native sequence. More preferably the variant has 10 mutations or less, most preferably 5 mutations or less over the whole native CTB sequence.

Mutant

As used herein, the terms “mutant” and “mutation” refers to any detectable change in genetic material, such as, for example, any DNA, or any process, mechanism or result of such a change. This includes gene mutations, in which the structure (such as the DNA sequence) of a gene is altered, any gene or DNA arising from any mutation process, and any expression product (such as, for example, RNA, protein or enzyme) expressed by a modified gene or DNA sequence. A mutant may arise naturally, or may be created artificially (such as, for example, by site-directed mutagenesis). Preferably the mutant has at least 90% sequence identity with the native or naturally occurring or wild type CTB sequence. Preferably the mutant has 20 mutations or less over the whole wild-type CTB sequence. More preferably the mutant has 10 mutations or less, most preferably 5 mutations or less over the whole wildtype CTB sequence.

By way of example, a CTB variant may include any subunit protein including at least one mutation, addition, or deletion of residues between positions 1-103 of CTB is disclosed. Examples of such mutations include any point mutation, deletion or insertion into these toxins, subunits or other proteins as well as any peptide extensions to these proteins whether placed in the amino-end, the carboxy-end or elsewhere in the protein and irrespective of whether these peptides have immunological properties by being B cell epitopes, T cell epitopes or otherwise which are capable of stimulating or deviating the immune response. For example, a number of such mutants have been described in the literature (Backstrom et al; Gene 1995; 165: 163-171; Backstrom et al., Gene 1996; 169: 211-217; Schodel et al., Gene 1991; 99: 255-259; Dertzbaugh et al. Infect. Immun. 1990; 58: 70-79).

Homology

As used herein, the term “homology” refers to the degree of similarity between x and y. The correspondence between the sequence from one form to another can be determined by techniques known in the art. For example, they can be determined by a direct comparison of the sequence information of the polynucleotide. Alternatively, homology can be determined by hybridization of the polynucleotides under conditions which form stable duplexes between homologous regions (for example, those which would be used prior to S1 digestion), followed by digestion with single-stranded specific nuclease(s), followed by size determination of the digested fragments.

Homologue

Any CTB sequences, such as but not limited to that presented in FIG. 14 or those described under their GI Accession Numbers in Table 1 may be useful in the present invention. In one embodiment, a CTB protein expressed by a V. cholerae host cell of the invention may be encoded by:
(i) a DNA molecule comprising the nucleotide sequence of the CTB gene presented in FIG. 14 or specified in Table 1 by GenBank accession number;
(ii) a DNA molecule which hybridises to the complement of the nucleotide sequence in (a); or
(iii) a DNA molecule which encodes the same amino acid sequence as the DNA molecule of (a) or (b) but which is a degenerate form of the DNA molecule of (a) or (b).

As defined herein, the term “homologue” refers to an entity having a certain homology with the native or wild type amino acid sequence and the native or wild type nucleotide sequence. Here, the term “homology” can be equated with “identity”. A homologue of the polynucleotide sequence in (i) may be used in the invention. Typically, a homologue has at least 40% sequence identity to the corresponding specified sequence, preferably at least 60%, 70%, 75%, 80% or 85% and more preferably at least 90%, 95% or 99% sequence identity. Such sequence identity may exist over a region of at least 15, preferably at least 30, for instance at least 40, 60 or 100 or more contiguous nucleotides. Typically, the homologues will comprise the same-active-sites and the like as the subject amino acid sequence. Although homology can also be considered in terms of similarity (that is, amino acid residues having similar chemical properties/functions), in the context of the present invention it is preferred to express homology in terms of sequence identity.

Methods of measuring polynucleotide homology are well known in the art. Homology comparisons can be conducted by eye, or more usually, with the aid of readily available sequence comparison programs. These commercially available computer programs can calculate percent homology between two or more sequences. Percent homology may be calculated over contiguous sequences. That is, one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this method is a very simple and consistent method, it fails to take into consideration that, for example, in an otherwise identical pair of sequences, one insertion or deletion will cause the following amino acid residues to be put out of alignment, thus potentially resulting in a large reduction in percent homology when a global alignment is performed. Consequently, most sequence comparison methods are designed to produce optimal alignments that take into consideration possible insertions and deletions without penalizing unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible reflecting higher relatedness between the two compared sequences—will achieve a higher score than one with many gaps. “Affine gap costs” are typically used that charge a relatively high cost for the existence of a gap and a smaller penalty for each subsequent residue in the gap.

This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al 1984, Nucleic Acids Research 12: 387-395). Examples of other software than can perform sequence comparisons include, but are not limited to, the BLAST package (see Ausubel et al 1999 ibid Chapter 18), FASTA (Atschul et al 1990, J. Mol. Biol., 403-410) and the GENEWORKS suite of comparison tools. Both BLAST and FASTA are available for offline and online searching (see Ausubel et al 1999 ibid, pages 7-58 to 7-60) and Altschul (1993) J Mol Evol 36: 290-300 or Altschul et al (1990) J Mol Biol 215: 403-10. However, for some applications, it is preferred to use the GCG Bestfit program. A new tool, called BLAST 2 Sequences is also available for comparing protein and nucleotide sequence (see FEMS Microbiol Lett 1999 174 (2): 247-50; FEMS Microbiol Lett 1999 177 (1): 187-8 and tatiana&commat;ncbi.nlm.nih.gov).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62. Once the software has produced an optimal alignment, it is possible to calculate percent homology, preferably percent sequence identity. The software typically does this as part of the sequence comparison and generates a numerical result.

The homologue may differ from the corresponding specified sequence by at least 1, 2, 5, 10 or more substitutions, deletions or insertions over a region of at least 30, for instance at least 40, 60 or 100 or more contiguous nucleotides, of the homologue. Thus, the homologue may differ from the corresponding specified sequence by at least 1, 2, 5, 10, 30 or more substitutions, deletions or insertions. A homologue CTB gene may be tested by expressing the gene in a suitable host and testing for cross reactivity with antibody specific to the particular CTB antigen.

The expression plasmid used in the present invention may comprise nucleotide sequences that can hybridise to the nucleotide sequences presented herein (including complementary sequences of those presented herein). A homologue typically hybridises with the corresponding specified sequence at a level significantly above background. The signal level generated by the interaction between the homologue and the specified sequence is typically at least 10 fold, preferably at least 100 fold, as intense as background hybridisation. The intensity of interaction may be measured, for example, by radiolabelling the probe, such as, for example, with ³²P.

Selective hybridisation is typically achieved using conditions of medium to high stringency, for example 0.03M sodium chloride and 0.003M sodium citrate at from about 50° C. to about 60° C. In a preferred aspect, the present invention covers nucleotide sequences that can hybridise to the nucleotide sequence of the present invention under stringent conditions (such as, for example, 65° C. and 0.1 SSC) to the nucleotide sequence presented herein (including complementary sequences of those presented herein).

Fragment

The term “fragment” indicates that the polypeptide comprises a fraction of the wild type amino acid sequence. It may comprise one or more large contiguous sections of sequence or a plurality of small sections. Preferably the polypeptide comprises at least 50%, more preferably at least 65%, most preferably at least 80% of the wild-type sequence.

Heterologous Protein/Molecule

In contrast to the poor immunogenicity of the A subunit alone, both LTB and CTB are exceptionally potent immunogens. Because of their immunogenicity, both LTB and CTB have been used as carriers for other epitopes and antigens (Nashar et al Vaccine 1993; 11(2):235-40) and have been used as components of vaccines against cholera and E. coli mediated diarrhoeal diseases (Jetborn et al 1992 Vaccine 10: 130).

The CTB produced by the expression system as described herein may also be used as a carrier for other immunogeneic or tolerogeneic molecules, such as heterologous molecules, which may be coupled to CTB by chemical conjugation or which may be prepared as part of a chimeric protein.

As used herein, the term “heterologous molecule” refers to a molecule which is typically from a different species to the host cell, but may be from a different or unrelated strain of the same species. The host cell may be engineered to express more than one heterologous polypeptide, in which case the polypeptides may be from the same organism or from different organisms. In a preferred embodiment of the invention, a heterologous nucleotide sequence encodes a heterologous antigen of a pathogen. In another preferred embodiment, two or more heterologous antigens from different pathogens may be expressed. The heterologous DNA or heterologous polypeptide may be a complete protein or a part of a protein containing an epitope. In one embodiment of the invention, the heterologous polypeptide may be the non-toxic component or form of CT or LT. In another embodiment, the heterologous antigen may be an ETEC antigen such as CFA1, CFAII (CS1, CS2, CS3), CFA IV (CS4, CS5, CS6) fimbrial antigen. In yet another embodiment, the heterologous antigen may be expressed or be prepared as part of a fusion protein. In this regard, the fusion protein may involve two or more different antigens or an antigen and a region designed to increase the immunogenicity of a heterologous polypeptide. The heterologous antigen may be selected from the group consisting of viruses, bacteria, fungi, proteins, polypeptides or immunogenic portions thereof. In another embodiment, the immunogenic component is selected from the group consisting of Bordetella pertussis toxin subunit S2, S3, S4, S5, Diphtheria toxin fragment B, E. coli fimbria K88, K99, 987P, F41, CFA I, CFA II (CS 1, CS2, CS3), CFA IV (CS4, CS5, CS6).

In some embodiments of the invention the heterologous polypeptide encoded by the plasmid to be stabilised may be other than, or in addition to, sequences encoding a heterologous antigen. For example, the polypeptide may regulate or turn on expression of the heterologous antigen encoded by sequences on the bacterial chromosome or a second plasmid. Alternatively, or in addition, the heterologous polypeptide encoded by the plasmid may be a selection marker or a polypeptide required for optimal growth of the bacterium carrying the plasmid.

In the case of the heterologous polypeptide playing a regulatory role it may bind to and activate, or increase expression from, the sequences encoding the heterologous antigen. The regulation may be inducible so that expression of the antigen is only activated at an appropriate time, for example when the bacteria are at an appropriate stage of growth or administered to the host to be vaccinated.

This may help avoid, or reduce, early selection pressure against bacteria carrying the plasmid until expression is induced.

As indicated above, one or more heterologous molecules may be coupled to the CTB produced by the expression system as described herein by chemical conjugation or which may be prepared as part of a chimeric protein. In one embodiment of the present invention, chemical coupling is carried out using a functional cross-linking reagent, such as a heterobifunctional cross-linking reagent. More preferably the cross-linking agent is N-y(-maleimido-butyroxyl)succinimide ester (GMBS) or N-succinimidyl-(3-pyridyl-dithio)-propionate (SPDP). The term “coupling” includes direct or indirect linkage, for example, by the provision of suitable spacer groups. By way of example, the coupled components may be covalently linked, to form a single active moiety/entity. Alternatively, the coupled components may also be linked to another entity. WO 95/10301 teaches how antigens may be coupled either directly or indirectly to a mucosa-binding molecule.

Method have also been described for making fusion proteins based on CTB or LTB wherein nucleic acids encoding for either or both of T or B epitopes of a heterologous antigen of interest are genetically fused to coding sequences for either or both of the N- or C-terminus of CTB, or placed in an intrachain position in the CTB or LTB coding sequence, or to analogous positions in CTA or LTA (Backstrom et al., Gene 1995; 165: 163-171, Bäckström et al., Gene 1994; 149: 211-217, Schödel et al., Gene 1991; 99: 255259). Methods have also been described for fusing peptides to the carboxy or amino ends of CTA or LTA and for co-expressing these fusion proteins with CTB or LTB (Sanchez et al. FEBS Lett. 1986; 208: 194-198, Sanchez et al. FEBS Lett. 1997; 401: 95-97).

By way of example, genetic fusions may be prepared using a vector that contains a promoter for expressing the fusion protein, the DNA sequence of the cholera toxin binding subunit CTB, and an immunogenic peptide coding sequence. The CTB and the immunogenic peptide coding sequence are linked such that they were in the proper reading frame producing a fusion protein. The fusion protein is expressed, secreted, and purified for use as a vaccine. Hybrid CTB/LTB proteins may also be prepared according to the teachings in WO 96/34893 or in accordance with any known method in the art. These expressed hybrid proteins may include a mature CTB sequence in which the amino acid residues are substituted with the corresponding amino acid residues of mature LTB which impart LTB specific epitopes characteristic to said immunogenic mature CTB (resulting in, for example, a hybrid molecule called LCTBA). Conversely, the hybrid protein may include a mature LTB sequence in which the amino acid residues are substituted with the corresponding amino acid residues of mature CTB which impart CTB specific epitopes characteristic to said immunogenic mature LTB (resulting in, for example, a hybrid molecule called LCTBB). In addition a third hybrid protein is envisaged which combines an LCTBA molecule and a LCTBB molecule (see WO 96/34893 and Lebens et al (1996) Infect and Immunity 64(6); 2144-2150).

Method of Making CTB

Examples of a gene encoding CTB include but are not limited to the CTB gene SEQ ID NO: 1 also presented in FIG. 14 and those specified under GI Accession No in Table 1. The CTB gene is inserted in an expression vector. The stable expression vector may be made and transformed into bacterium using conventional techniques.

As used herein, the term “transformation”, refers to the insertion of an exogenous polynucleotide into a host cell, heterologous gene, nucleotide sequence, such as a DNA or RNA sequence so that the host cell will express the introduced gene or sequence which is typically an RNA coded by the introduced gene or sequence, but also a protein or an enzyme coded by the introduced gene or sequence. Any method may be used for the insertion such as but not limited to direct uptake, transduction, f-mating, use of CaCl₂ or other agents, such as divalent cations and DMSO or electroporation. The heterologous or exogeneous polynucleotide may be maintained as a non-integrated vector, for example, a plasmid, or alternatively, may be integrated into the host genome.

Transformation procedures usually vary with the bacterial species to be transformed. See e.g., [Masson et al. (1989) FEMS Microbiol. Lett. 60:273; Palva et al. (1982) Proc. Natl. Acad. Sci. USA 79:5582; EPO Publ. Nos. 036 259 and 063 953; PCT Publ. No. Wo 84/04541, Bacillus], [Miller et al. (1988) Proc. Natl. Acad. Sci. 85:856; Wang et al. (1990) J. Bacteriol. 172:949, Campylobacter], [Cohen et al. (1973) Proc. Natl. Acad. Sci. 69:2110; Dower et al. (1988) Nucleic Acids Res. 16:6127; Kushner (1978) “An improved method for transformation of Escherichia coli with ColE1-derived plasmids. In Genetic Engineering: Proceedings of the International Symposium on Genetic Engineering (eds. H. W. Boyer and S. Nicosia); Mandel et al. (1970) J. Mol. Biol. 53:159; Taketo (1988) Biochim. Biophys. Acta 949:318; Escherichia], [Chassy et al. (1987) FEMS Microbiol. Lett. 25 44:173 Lactobacillus]; [Fiedler et al. (1988) Anal. Biochem 170:38, Pseudomonas]; [Augustin et al. (1990) FEMS Microbiol. Lett. 66:203, Staphylococcus], [Barany et al., (1980) J. Bacteriol. 144:698; Harlander (1987) “Transformation of Streptococcus lactis by electroporation, in: Streptococcal Genetics (ed. J. Ferretti and R. Curtiss III); Perry et al., (1981) Infec. Immun. 32:1295; Powell et al. (1988) Appl. Environ. Microbiol. 54:655; Somkuti et al. (1987) Proc. 4th Evr. Cong. Biotechnology 1:412, Streptococcus].

A host cell that receives and expresses introduced DNA or RNA has been “transformed” and is a “transformant” or a “clone”. The DNA or RNA introduced to a host cell can come from any source, including cells of the same genus or species as the host cell or cells of a different genus or species.

Means for introducing the stable expression vector into prokaryotic host cells, such as V. cholerae host cells are known in the art. Examples of suitable methods include but are not limited to electroporation, conjugation and electrophoresis. The transformed colonies may be screened and selected for correct uptake using standard screening and selection procedures. The expression of the CTB is designed so that CTB is overproduced and accumulates in the growth medium.

After culturing, the CTB subunit protein produced by the expression system of the present invention as described herein may be purified by, for example, chromatography, precipitation, and/or density gradient centrifugation. The thus obtained CTB protein may be used as a vaccine or for the production of antibodies directed against said peptides, which can be used for passive immunization.

The CTB produced by the expression system as described herein may be purified from the culture filtrate using standard ammonium sulphate precipitation, ion-exchange and affinity chromatography techniques (as outlined in WO 01/27144). The CTB is characterised using GM-1 ELISA, calorimetric protein assays (A280, Lowry, Bradford, BCA), Western Blots and Single radial immunodiffusion (SRI) and Mancini test (as described in the Examples). Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Isolated CTB

The isolated stably expressed CTB obtainable by the method as described herein is essentially free of antibiotic residues because the expression system does not express an antibiotic resistance marker and therefore the use of antibiotic additives in the expression system is necessary.

In one embodiment, the V. cholerae host cell expresses at least one heterologous antigen.

In another embodiment, the V. cholerae host cell expresses a number of different antigens so that the Vibrio cholerae host cell is multivalent.

### EXAMPLES

The present invention is also described by means of examples, including the particular Examples presented here below in which reference is made to the following Figures. The use of such examples anywhere in the specification is illustrative only and in no way limits the scope and meaning of the invention or of any exemplified term.

### BRIEF DESCRIPTION OF THE DRAWINGS

FIG. 1 shows the cloning of a 1.4 kb EcoRI/HindIII fragment in pUC19;

FIG. 2 shows the insertion of a KanR-resistance gene block in the PstI site of the V. cholerae thyA gene in pUC19;

FIG. 3 shows the PCR primers used to generate thyA-Kan fragment with XbaI ends;

FIG. 4 shows the insertion of the thyA Kan fragment into XbaI restricted pNQ 705;

FIG. 5 shows the elimination of the start of the coding region of the Kanamycin gene and part of the thyA gene;

FIG. 6 shows the insertion of the ΔthyA ΔKan fragment into XbaI restricted pDM4;

FIG. 7 shows the PCR amplification and subcloning of the E. coli thyA gene in pUC19;

FIG. 8 shows the generation of pMT-thyA/cat;

FIG. 9 shows the insertion of the eltb-ctxB coding fragment from pML-LCTBλ2 in pMT-thyA/cat;

FIG. 10 shows the insertion of tac promotor in pMT-thyA/cat(ctxB) and the generation of pMT-ctxB/thyA(cat);

FIG. 11 shows the removal of the cat gene, generation of pMT-ctxB/thyA;

FIG. 12 shows the PCR reaction to remove superfluous V. cholerae DNA from pMT-ctxB/thyA, generation of pMT-ctxBthyA-2;

FIG. 13 is a graphic representation of the parts of pMT-ctxBthyA-2 that has been sequenced on Master Seed lot and consistency batches;

FIG. 14 presents the DNA sequence of the expression plasmid pMT-ctxBthyA-2; (204-295: E. coli thyA coding region; 1192-1876: Col E1 origin of replication; 2339-2710: eltB-ctxB coding region; 2402-2710: ctxB coding region; and 2732-2759: trpA terminator).

TABLE 1
Some CTB/LTB sequences

| Protein | NCBI Accession No |
|---|---|
| CTB | GI: 209555 |
| | GI: 433859 |
| | GI: 48420 |
| | GI: 48888 |
| | GI: 155296 |
| | GI: 48347 |
| | GI: 758351 |
| | GI: 1827850 |
| | GI: 808900 |
| | GI: 229616 |
| | GI: 998409 |
| | GI: 2144685 |
| | GI: 1421511 |
| CTB classic (596B) | GI: 48890 |
| CTB Ogawa 41 | GI: 2781121 |
| CTB Ogawa 41 (R35D) | GI: 1421525 |
| Classic LTB | GI: 3062900 |
| | GI: 1169505 |
| | GI: 1395122 |
| | GI: 145833 |
| LT 87 | GI: 1648865 |
| | GI: 223254 |
| | GI: 408996 |
| | GI: 494265 |
| | GI: 69630 |
| LT-IIa | GI: 146671 |
| LT-Iib | GI: 152784 |

TABLE 2
Some Leader/Signal sequences

| Endotoxin Signal | |
|---|---|
| Sequence | Signal sequence |
| LTB signal sequence | MNKVKCYVLFTALLSSLCAYG |
| | (SEQ ID NO: 5) |
| CTB V. cholera | MNKVKFYVLFTA LLSS |
| classic strain | LCAH GAPGYAHG |
| 569B CTB gene | (SEQ ID NO: 6) |
| signal sequence | |
| LTB Signal sequence | MNKVKFYVLFTA LLSS |
| for “401” strain of | LCAH G = 2laa |
| the present | (SEQ ID NO: 2) |
| invention | |

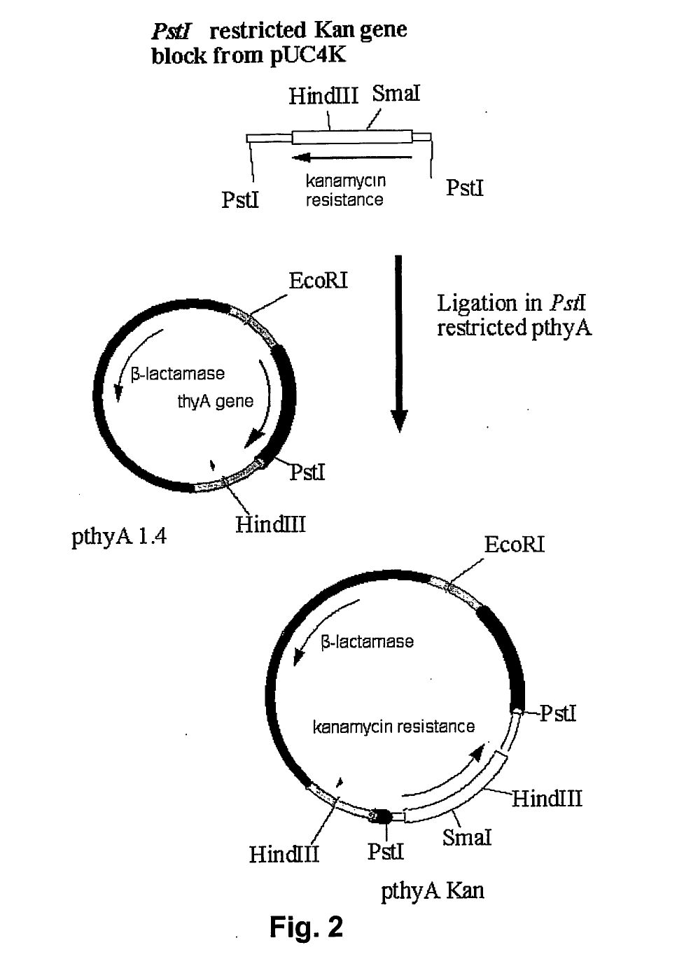

TABLE 3
Comparison between known CTB plasmids and the CTB plasmid described herein

| | | | Plasmid | | | |
|---|---|---|---|---|---|---|
| | | | Selection | | | |
| Plasmid | Reference | Host cell | Marker | Plasmid size | CTB sequence | Yield of CTB |
| PJS162 | Sanchez and | Toxin deleted | Ampicillin | About 10.2 kb | LTB leader sequence CTB | 0.04-0.05 mg/ml or |
| (pJS213) | Holmgren (1989) | JBK70 V. | resistance | | coding sequence CTB | 0.05-0.1 mg/ml (see |
| | | cholerae | marker (AmpR) | | genomic sequence (down | p482, col 2) |
| | | | | | stream of CTB gene) | |
| PML358 | Lebens et al | Rifampicin | Ampicillin | Plasmid size | LTB leader sequence CTB | 1 mg/ml when |
| | (1993) | Resistant | resistance | not disclosed | coding sequence CTB | ampicillin was |
| | | CtxA deleted | marker (AmpR) | | genomic sequence (down | maintained in the |
| | | derivative of | | | stream of CTB gene) | growth medium |
| | | classical 569B | | | | |
| | | V. cholerae | | | | |
| | | JS1569 strain | | | | |
| PNU212-CTB | Ichikawa et al | Bacillus | Erythromycin | About 4.8 kb | Promoter and signal | 1.4 mg/ml after a |
| | (1993) FEMS | Brevis | resistance | | sequence for one of the | period of 4-5 days |
| | Microbiol Lett | | gene (EmR) | | major extracellular | with antibiotics |
| | 111: 219-224 | | | | proteins (MWP) of | present in the |
| | | | | | B Brevis used with CTB | growth medium |
| | | | | | sequence | |
| PML-CTBtac1 | WO 01/27144 | Bacterial | Kanamycin | About 3.66 kb | CTB leader sequence CTB | Five times the |
| | (Active Biotech | host strain | resistance | | coding sequence CTB | product generated |
| | AB) (page 46-47 | | marker | | genomic sequence (down | by pJS162 (see page |
| | of app as filed | | | | stream of CTB gene) | 46, I32-34) (0.8 |
| | and FIG. 2) | | | | | mg/ml - see page |
| | | | | | | 47, lines 12-13) |
| PJS752-3 | | V. cholerae | Ampicillin | About 5.75 kb | LTB leader sequence CTB | About 0.4 mg/ml |
| | | | resistance | | coding sequence CTB | (see yield table) |
| | | | marker (AmpR) | | genomic sequence (down | |
| | | | | | stream of CTB gene) | |
| PMT- | | V. cholerae | thyA (non | About 2.8 kb | LTB leader sequence CTB | About 1.4 mg/ml |
| ctxBthyA-2 | as described | | antibiotic | | coding sequence (about | (see yield table) |
| | herein | | selection | | 800 bp removed | after only 18 hours |
| | | | marker) | | downstream of CTB | |
| | | | | | coding sequence) | |

### Example I

A. Source Materials

The origin of the ctxB gene is the V. cholerae serotype O1 strain 395 (Ogawa) [2]. The eltB signal sequence was obtained from plasmid pMMB68 [3]. The ligation of the eltB and ctxB gene is described in [4].

The origin of replication is ColE1 obtained from pBlueScript KS⁻ (Stratagene).

The origin of the tac promotor used in pMT-ctxBthyA-2 is from plasmid pKK223-3 (Pharmacia).

The DNA sequence used to PCR amplify the E. coli thyA gene was E. coli SY327 [5].

The Kan^{R} resistance gene block used in inactivation of the chromosomal V. cholerae thyA locus was obtained from pUC4K (Pharmacia).

The suicide vectors used for site-directed mutagenesis of the V. cholerae thyA locus were pNQ705 [6] and pDM4 described in [7].

The sequence of the V. cholerae thyA gene was determined at SBL Vaccin AB and is published in EMBL/Genebank under accession No AJ006514.

A.1Construction of the V. Cholerae Inaba Strain 401 Classical Biotype for Production of rCTB.

A. 1.2. Construction of the Host Strain V. Cholerae JS1569 ΔthyAΔKan.

The V. cholerae strain JS1569 ΔthyAΔkan is a classical O1 rifampicin resistant cholera strain originally derived from V. cholerae strain 569B; ATCC No 25870. The two copies of the cholera enterotoxin genes have been deleted by site-directed mutagenesis The attenuation comprises of a deletion of the cholera toxin A subunit gene.

[9]. The deletion and insertional inactivation of the thyA gene is described below.

A. 1.3. Inactivation of the thyA Gene in V. Cholerae JS1569.

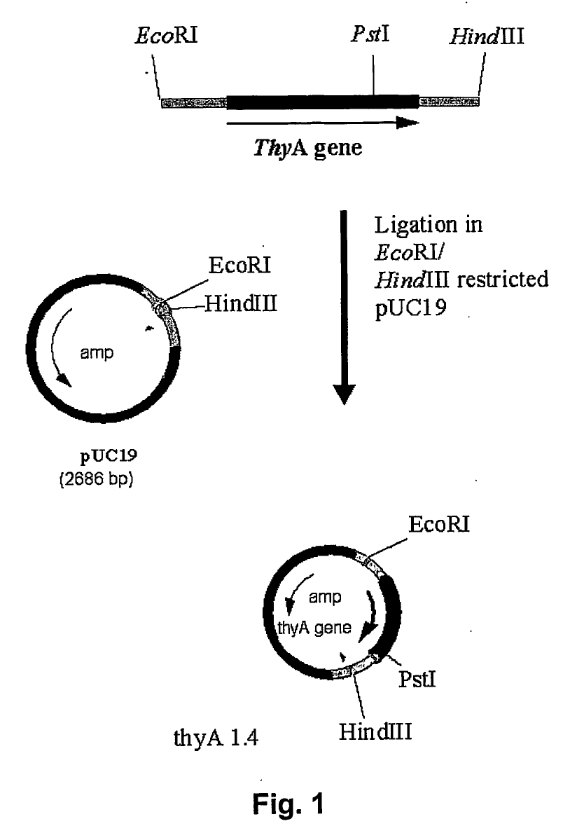

In the course of isolation and sequencing of the thyA gene from JS1569 [Carlin et al, Genebank accession no AJ006514) an EcoRI-HindIII fragment encompassing the entire thyA gene was cloned in the vector pUC19 on a 1.4 kb DNA fragment. This plasmid is called thyA 1.4 (FIG. 1.).

A. 1.4 Inactivation of the thyA Gene by Insertion of a Kan^{R} Gene Block.

The plasmid pthyA1.4 was cleaved with PstI, and ligated to a PstI fragment of the Kan^{R} gene block from pUC4K (Pharmacia Biotech). The ligation mixture was electroporated into E. coli HB101 and transformants were selected for by plating on Syncase agar plates containing ampicillin and kanamycin. This plasmid is called pthyA Kan. (FIG. 2).

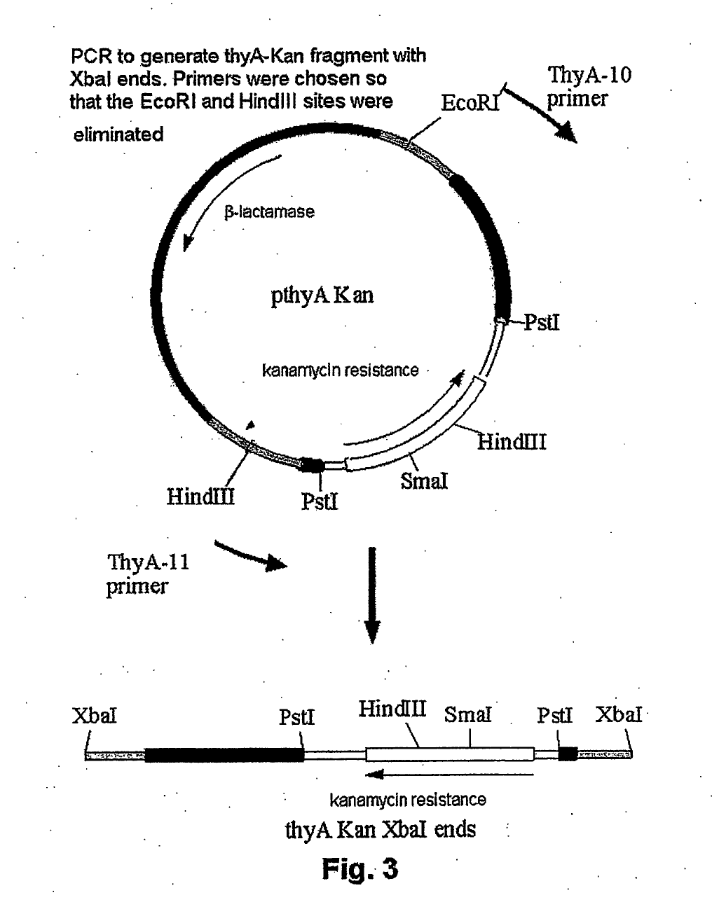

The thyA Kan^{R} gene was PCR amplified from pthyA Kan using a mix of Taq and Pwo DNA polymerases which yields PCR fragments with high sequence fidelity (Expand™ High fidelity PCR system, Boehringer Mannheim) The primers used were thyA-10 GCT CTA GAG CCT TAG MG GCG TGG TTC (SEQ ID NO: 7) and thyA-11 GCT CTA GAG CTA CGG TCT TGA TTT ACG GTA T (SEQ ID NO: 8) generating a PCR fragment with XbaI ends (FIG. 3).

This fragment was digested with XbaI and ligated into the vector pMAL-C2 that had been digested with XbaI and dephosphorylated as indicated above. The fragment size and orientation was confirmed by restriction enzyme analysis.

A. 1.5 Insertional Inactivation of the thyA Gene in the V. Cholerae Chromosome by Site-Directed Mutagenesis.

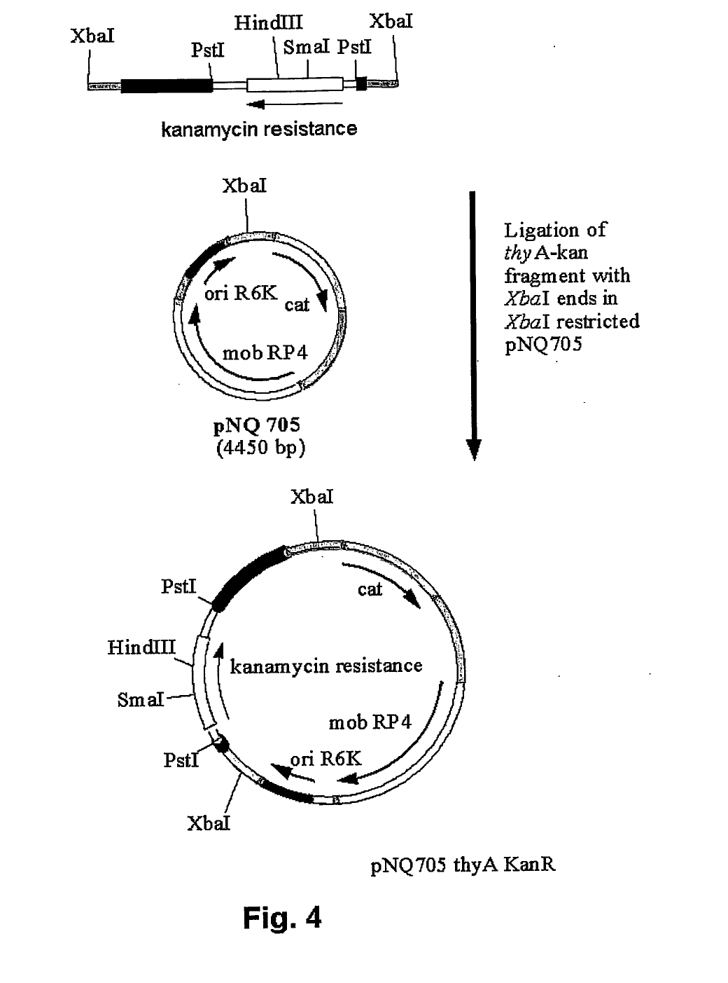

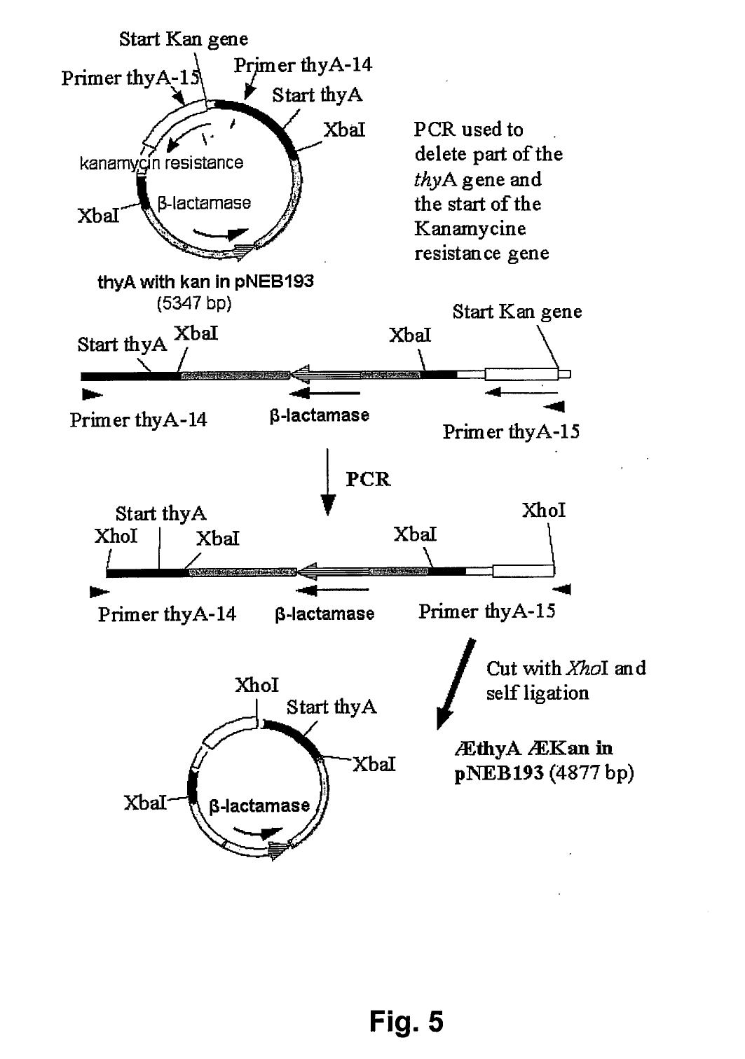

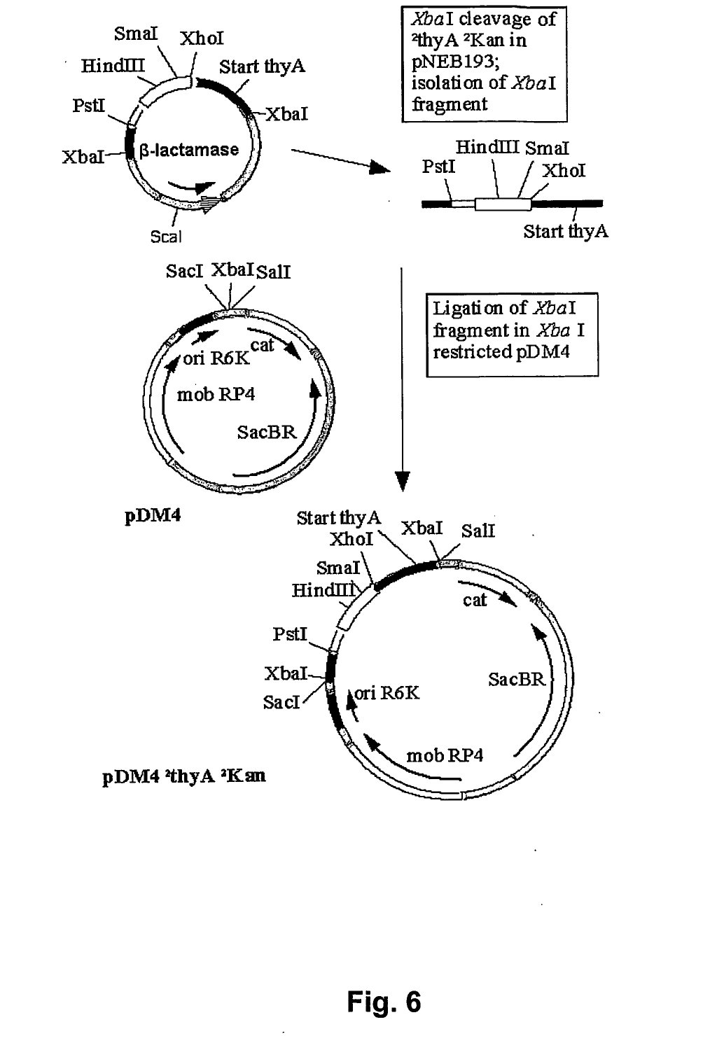

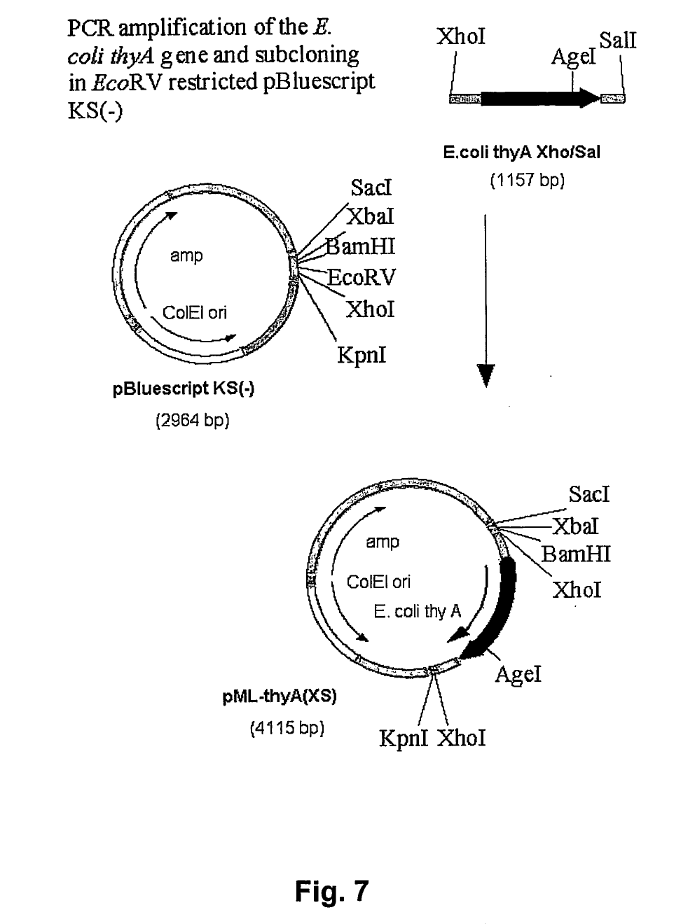

The suicide vector pNQ705 [6] (FIG. 4) contains the R6K origin of replication and hence has to be maintained in a host harbouring the pir gene. It also contains the mobRP4 genes and a CAT gene allowing for chloramphenicol selection.

The thyA Kan^{R} gene was excised from pMAL-C2 as a XbaI fragment and ligated into XbaI digested pNQ705 (FIG. 4.) The ligation mix was electroporated into E. coli SY327 [Δ(lac pro) argE(Am) rif malA recA56] and transformants were selected for on plates containing chloramphenicol. Restreaked individual colonies were analysed with restriction enzymes for presence and orientation of insert.

The resulting plasmid pNQ705 thyA Kan^{R} was transformed by electroporation into E. coliS17-1 (thi pro hsdR hsdM⁺ recA RP4-2-Tc::Mu-Km::Tn7).

The mating between JS1569 thyA⁻ (a trimetoprim resistant variant of JS1569 carrying a single point mutation in the thyA gene [10] and E. coli S17-1 (pNQ705 thyA Kan^{R}) were done as streak matings on LB agar supplemented with rifampicin (50 μg/ml), Thymine (200 μg/ml) and Kanamycin (50 μg/ml) at 37° C. Individual colonies arising from the conjugation were transferred to liquid LB broth with rifampicin, thymine and Kanamycin supplements as above and were passaged for three days in this medium.

Transconjugants were at this time tested in PCR for insertion of the thyA Kan^{R} gene. Cultures that had the expected PCR fragment were plated out on LB agar supplemented as above.

Individual colonies were now picked and tested for sensitivity to chloramphenicol (25 μg/ml) and resistance to Kanamycin. These colonies were restreaked and individual colonies frozen. Phenotypically this strain was thymine dependent for growth.

A.1.6. Insertional Inactivation of the Kan^{R} Gene and Deletion of the thyA Gene.

An experiment was designed to replace the functional Kan^{R} gene with a truncated nonfunctional version and to remove a substantial part of the thyA gene to further ensure the stability of the thymine dependence in this strain. For this experiment the thyA Kan fragment with XbaI ends was subcloned Into XbaI digested pNEB 193 (New England Biolabs). Two PCR primers were designed thyA-14 and thyA-15 that both included XhoI cleavable ends. The primers were designed so as to eliminate 209 basepairs in the thyA gene and to eliminate 144 basepairs in the Kan^{R} gene (totaling 266 bp from the Kan^{R} gene block. The deletion in the Kan^{R} gene included the first 48 aminoacids of the Kan^{R} gene which would make successful transconjugants Kan^{s}. The resulting PCR fragment was cleaved with XhoI and allowed to selfligate, transformed into E. coli and transformants were selected for on ampicillin containing agar. Colonies were tested for Kanamycin sensitivity and the deletion was confirmed with restriction enzyme analysis.

From this plasmid the resulting ΔthyA ΔKan fragment was excised as an XbaI fragment. This fragment was inserted into the suicide vector pDM4 that had been digested with XbaI. pDM4 is derived from pNQ705 by replacing the multicloning site and insertion of the SacB gene from Bacillus subtitlis. The SacB gene encodes levansucrase gene that is lethal to Gram negative bacteria. The ligation mixture was transformed into E. coli SY327 and transformants were selected for by chloramphenicol. The insert size and orientation was verified by restriction enzyme analysis. For mating experiments the plasmid pDM4 ΔthyAΔKan was transformed into E. coli S-17. Mating was performed between E. coli S-17 (pDM4 ΔthyA Δ Kan) and JS1569 ΔthyA Kan on LB plates containing rifampicin, chloramphenicol and thymine. Transconjugants were grown further in LB broth with rifampicin, chloramphenicol and thymine. After passaging for three days in this medium colonies were plated on LB plates containing thymine and 5% sucrose. Emerging colonies on these plates were tested by replica plating for growth on medium with and without thymine, thymine plates containing chloramphenicol and plates containing kanamycin.

Chloramphenicol and kanamycin sensitive colonies with a requirement for thymine were selected and tested in PCR with appropriate primers for a replacement of the thyA Kan^{R} gene block with the ΔthyA ΔKan insert. Single colonies of this strain were restreaked. For these colonies re-confirmation of the genotype (rifampicin resistance, deletion of ctxA loci, thymine dependence, chloramphenicol, and kanamycin sensitivity) was done and the strain was named JS1569 ΔthyA ΔKan.

Further characterisation involved PCR amplification and partial sequencing of the modified chromosomal thyA locus in this strain. It was found that the point mutation in the trimetoprim resistant thyA⁻ strain used for the first mating experiment had been changed to wildtype i.e. the thymine dependence of this strain is caused by the deletion of the thyA gene further downstream. DNA sequencing also confirmed the deletion of the thyA and Kan gene block (data not shown).

B. The Expression Plasmid pMT-ctxBthyA-2

B.1.1. Cloning of the E. coli thyA Gene.

For the cloning of the E. coli thyA gene the published sequence (Genebank accession no J01709) was used to design PCR primers

(SEQ ID NO: 9)

MLthyA-1: ^{5′} GGG GGC TCG [symbol] G TTT GTT CCT GAT TGG
TTA CGG^{3′}

Letters in bold indicate sequence from the published sequence (bases 16-39 on the sense strand), italic letters indicate a XhoI site added to the sequence.

(SEQ ID NO: 10)

MLthyA-2: ^{5′} GGG GGG TCG ACG TTT CTA TTT CTT CGG
CGC ATC TTC^{3′}

Letters in bold indicate sequence from the published sequence (bases 1152-1128 on the non-sense strand), italic letters indicate a Sail site added to the sequence.
These primers were used to amplify the thyA gene from E. coli SY327.

The resulting PCR fragment was blunt-end repaired with T4 polymerase and cloned into pBluescript KS⁻ (Stratagene) in the EcoRV site in this vector. The ligated plasmid was transformed into E. coli XL1-Blue (Stratagene). Transformants were selected for on LB plates supplemented with ampicillin on the basis of blue/white colonies in the presence of X-Gal and IPTG. The inserted fragments size and orientation was confirmed by restriction enzyme cleavage. The functionality of the thyA gene was confirmed by electroporating the recombinant plasmid into JS1569 thyA and selection both for ampicillin resistance and growth on modified syncase medium in the absence of thymine. This plasmid is called pML-thyA(XS).

B. 1.2. Generation of a Cloning Vector Carrying the E. coli thyA Gene.

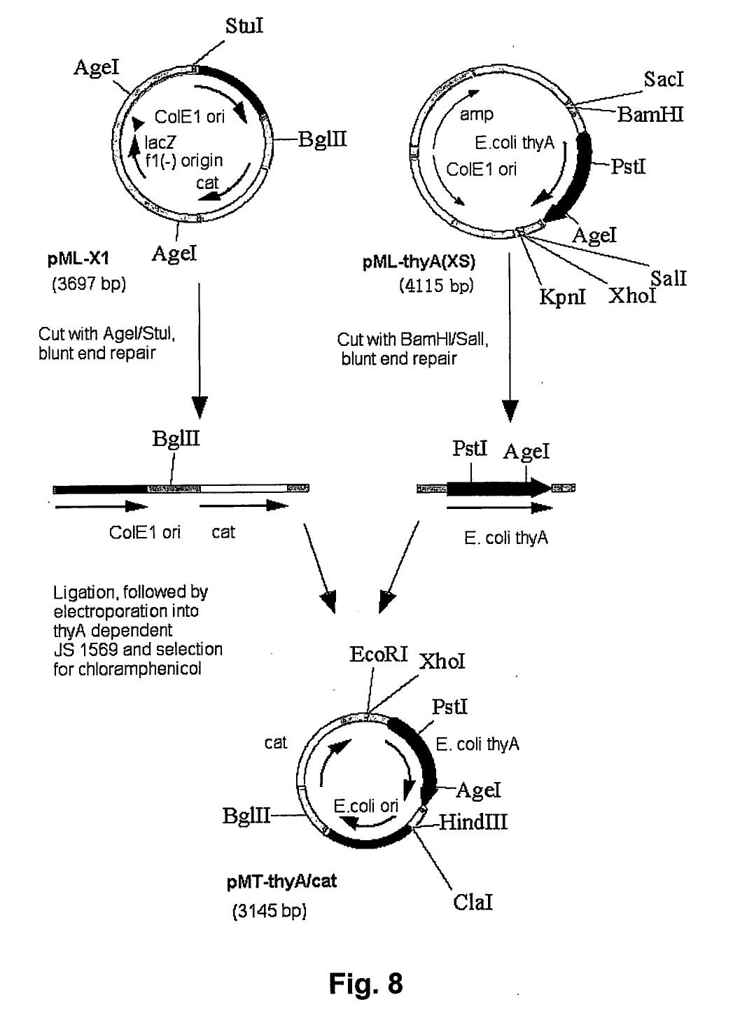

For the cloning of the E. coli thyA gene the vector pML-X1 was used (FIG. 8.) This plasmids origin is pBC SK⁻ (Stratagene). In pML-X1 the origin of replication (ColE1) is flanked by unique BglII and StuI sites and it also carries the chloramphenicol (cat) gene from pBC SK⁻ (FIG. 8).

pML-X1 DNA was digested with AgeI/StuI restriction enzymes and blunt-end repaired with T4 polymerase.

The pML-thyA(XS) vector was digested with BamHI/SalI and also blunt-end repaired (FIG. 8.) The two DNA preparations were mixed and ligated. The ligation mix was electroporated into JS1569 4.4 (A trimetoprim resistant variant of JS1569 carrying a single point mutation in its thyA gene) and thymine dependence and chloramphenicol resistance were selected for. The resulting plasmid pMT-thyA/cat was restreaked and subjected to extensive restriction enzyme analysis to verify the size and orientation of its different components (FIG. 8.)

B. 1.3. Insertion of ctxB into pMT-thyA/cat.

In order to insert the desired ctxB gene into the pMT-thyA/cat plasmid a 1.2 kb EcoRI/XhoI fragment from plasmid pML-LCTBλ2 was obtained. The plasmid pML-LCTBλ2 has been described earlier [4], briefly the ctxB gene was isolated from the plasmid pCVD30 [2]. The ctxB gene contained in the pCVD30 plasmid [2] originates from the V. cholerae serotype O1 strain 395 (Ogawa).

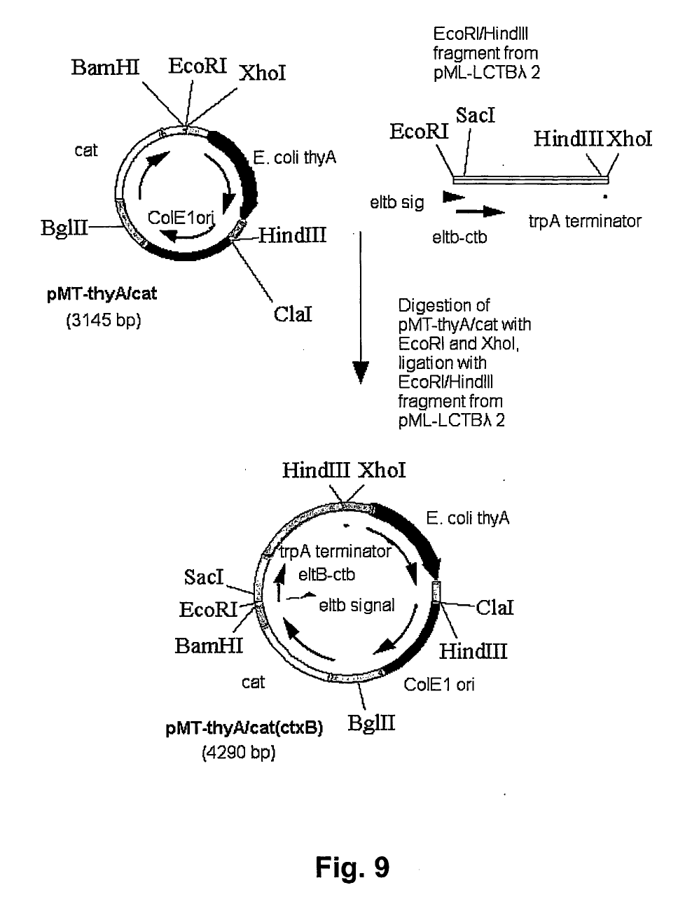

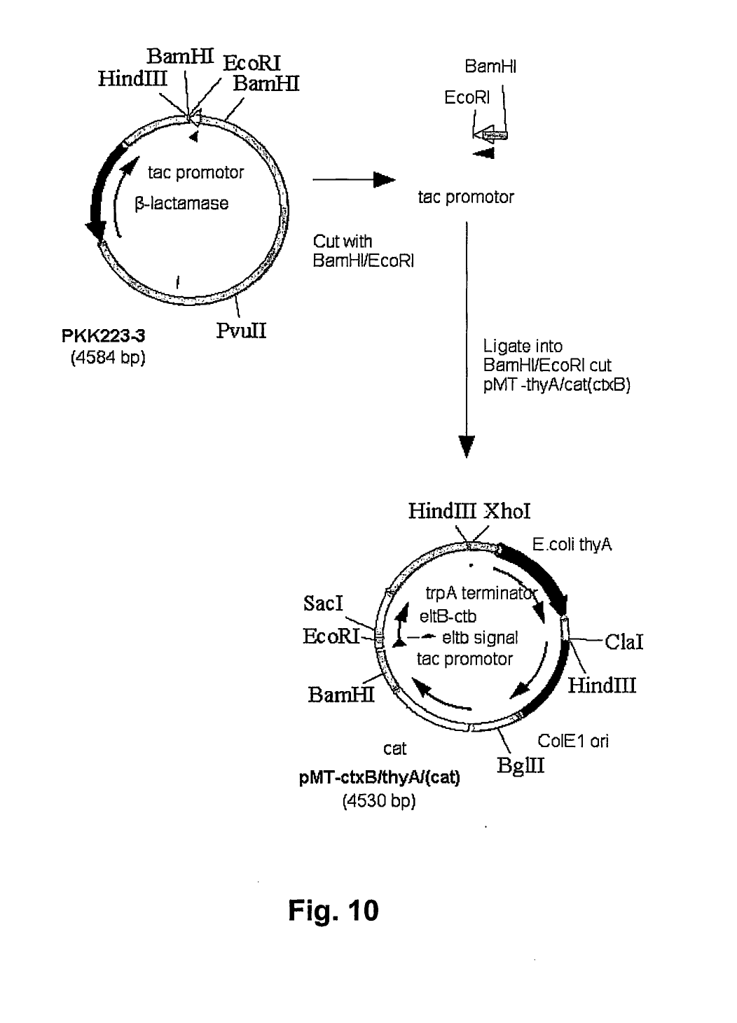

The ctxB gene in pML-LCTBλ2 is upstreams fused to the eltB signal peptide from the heat-labile enterotoxin of E. coli in such a way that a naturally occurring SacI site could be used (FIG. 9.). This also introduces an Alanine as the N-terminal amino acid rather than the naturally occurring Threonine. This modification of the N-terminal sequence and signal peptide has led to that only a single N-terminal sequence is formed from this new expression plasmid for rCTB as compared to the previously used pJS752-3 (see section C.2 below). Downstream of the ctxB gene on the EcoRI/XhoI fragment from pML-LCTBλ2 the powerful trpA terminators are located, effectively terminating m-RNA transcription. The EcoRI/XhoI fragment from pML-LCTBλ2 was ligated into the pMT-thyA/cat plasmid that had been digested with the same enzymes (FIG. 10), resulting in the plasmid pMT-thyA/cat(ctxB), which lacks a promotor upstream of the eltb signal peptide.

B. 1.4. Insertion of the tac Promotor into pMT-thyA/cat(ctxB).

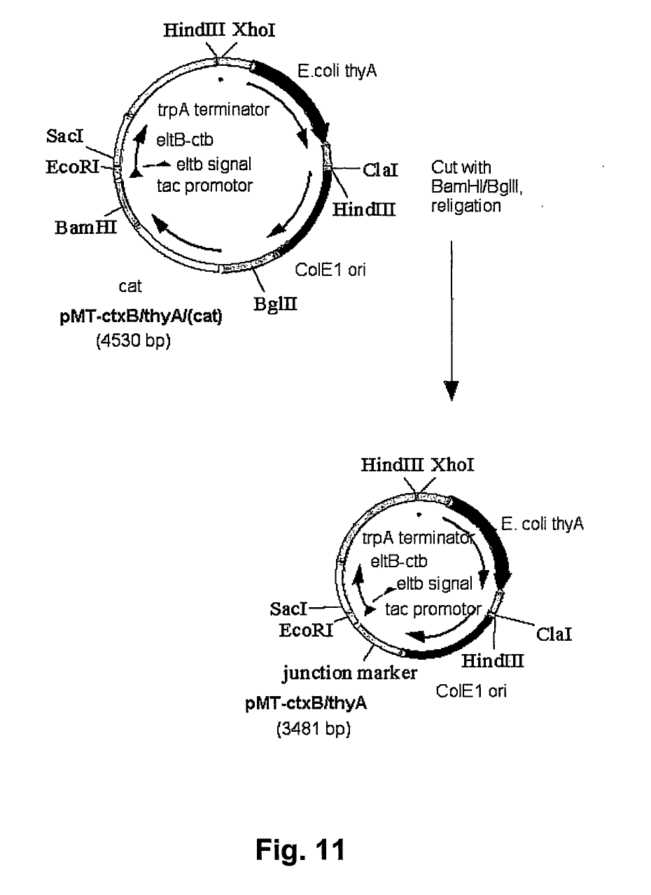

The tac promotor was inserted as a 256 base-pair BamHI/EcoRI fragment originally obtained from the cloning vector pKK223-3 (Pharmacia) (FIG. 11). Ligated DNA from this reaction was introduced into V. cholerae JS1569 4.4 and colonies were selected for on the basis of growth in the absence of thymine and resistance to chloramphenicol.

Transformants were screened both by restriction enzyme analysis of the recombinant plasmids and the production of CTB. Single colonies with the highest rCTB production as judged by GM1-ELISA were selected. The recombinant plasmid was named pMT-ctxB/thyA(cat).

B.1.5. Removal of the cat Gene.

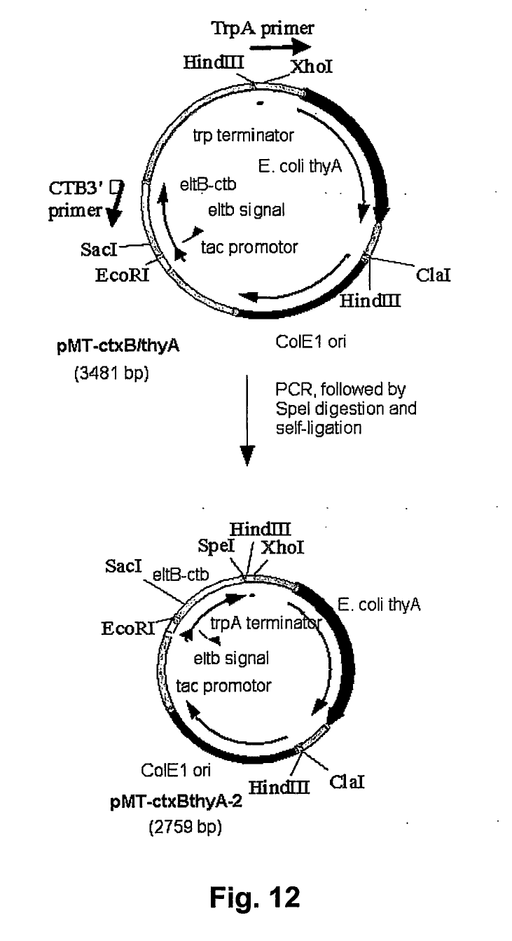

To remove the cat gene i.e. to obtain an expression plasmid without any antibiotic selection marker the pMT-ctxB/thyA(cat) plasmid was digested with the restriction enzymes BamHI and BglII. The cut plasmid was religated and again electroporated into the V. cholerae strain JS1569 4.4. Transformants were selected on the basis of growth in the absence of thymine and in the absence of chloramphenicol. Individual colonies were screened for sensitivity to chloramphenicol and checked for presence of plasmid in Wizard Miniprepps. Plasmids were analysed with restriction enzymes. Culture supernatants from these colonies were subjected to GM1 ELISA. The resulting plasmid was pMT-ctxB/thyA (FIG. 12).

B. 1.6. Removal of Superfluous V. Cholerae DNA from pMT-ctxB/thyA, Generation of pMT-ctxBthyA-2.

The EcoRI/XhoI fragment from pML-LCTBλ2 consists of approx 1200 base-pairs, of these only about 400 base-pairs code for the ctxB gene. There is in one reading frame going in the other direction of ctxB an open reading frame possibly coding for an orfF protein in the pyrF operon. The sequence is incomplete and thus probably not expressed. In order to remove the non-coding CTB portion, PCR primers were designed, the first so as to include the end of the ctxB gene (in italics below) and a SpeI site (in bold below). The other PCR primer was designed to include the trpA terminators (in italics below) and also a SpeI site (in bold below).

(SEQ ID NO: 11)

CTB3′:^{5′}GGG GGA CTA GTT TAA TTT GCC ATA CTA ATT
GCG GCA ATC G^{3′}

(SEQ ID NO: 12)

TrpA term:^{5′}GGG GGA CTA GTC AAT TGA AGC TTA AGC
CCG CCT AAT GAG CG³

The pMT-ctxB/thyA plasmid served as template for the PCR reaction. After obtaining an PCR fragment of the correct size, this was gel-purified, and digested with SpeI. The plasmid was allowed to self-ligate and was electroporated into V. cholerae JS1569 4.4. Transformants were selected on the basis of growth in the absence of thymine, single colonies isolated, restreaked, and plasmid DNA from these cultures was analysed by restriction enzyme analysis. Approximately 800 base-pairs of DNA including the entire orfF coding sequence was removed. GM1 ELISA indicated that this had no effect on the expression of CTB.

The resulting plasmid was called pMT-ctxBthyA-2 and is the final construct used in conjunction with the host strain V. cholerae JS1569 ΔthyAΔkan to form the rCTB producing strain V. cholerae strain 401.

B.1.7. Insertion of pMT-ctxBthyA-2 in V. Cholerae JS1569 ΔthyAΔkan.

The plasmid preparation from pMT-ctxBthyA-2 in V. cholerae JS1569 4.4 was electroporated into V. cholerae JS1569 ΔthyAΔkan thereby forming the strain V. cholerae Inaba strain 401 classical biotype. Transformants were selected for by their ability to grow in the absence of thymine. Individual colonies were tested for their ability to produce rCTB by colony lifts on nitrocellulose filters (SBL test method PT00020 as described below) using a monoclonal antibody specific for both CTB and LTB (E. coli heat-labile enterotoxin). Colonies were re-streaked to obtain single colonies and cultures from these colonies were used for plasmid analysis and extensive restriction analysis and finally frozen.

SBL Test Method PT00020

This is a method which is used to distinguish colonies of the rCTB producing strain that can produce rCTB from those that have lost that capacity. The methodology used consists of growing the bacterial colonies to be tested on an agar plate, transferring the colonies to a nitrocellulose filter. This filter is incubated with a monoclonal antibody specific for pentameric rCTB, washed and then incubated with an anti mouse IgG alkaline phosphatase conjugate. After washing the filters are developed with a precipitating dye, leaving the rCTB colonies bluish-black while non-producing colonies are left essentially colourless.

C. Description of the V. cholerae JS1569 ΔthyA Δkan Strain Carrying the pMT-ctxBthyA-2 Expression Vector: Strain V. cholerae 401.

C.1. Nucleotide Sequence of the ctxB Gene and Amino Acid Sequence of the Translated Polypeptide.

C1.1. Detailed Nucleotide Sequence of the ctxB Gene and Flanking Regions.

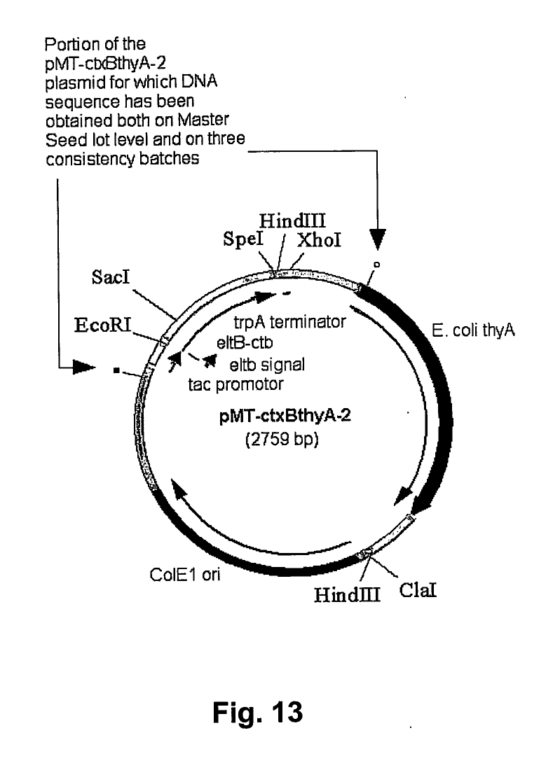

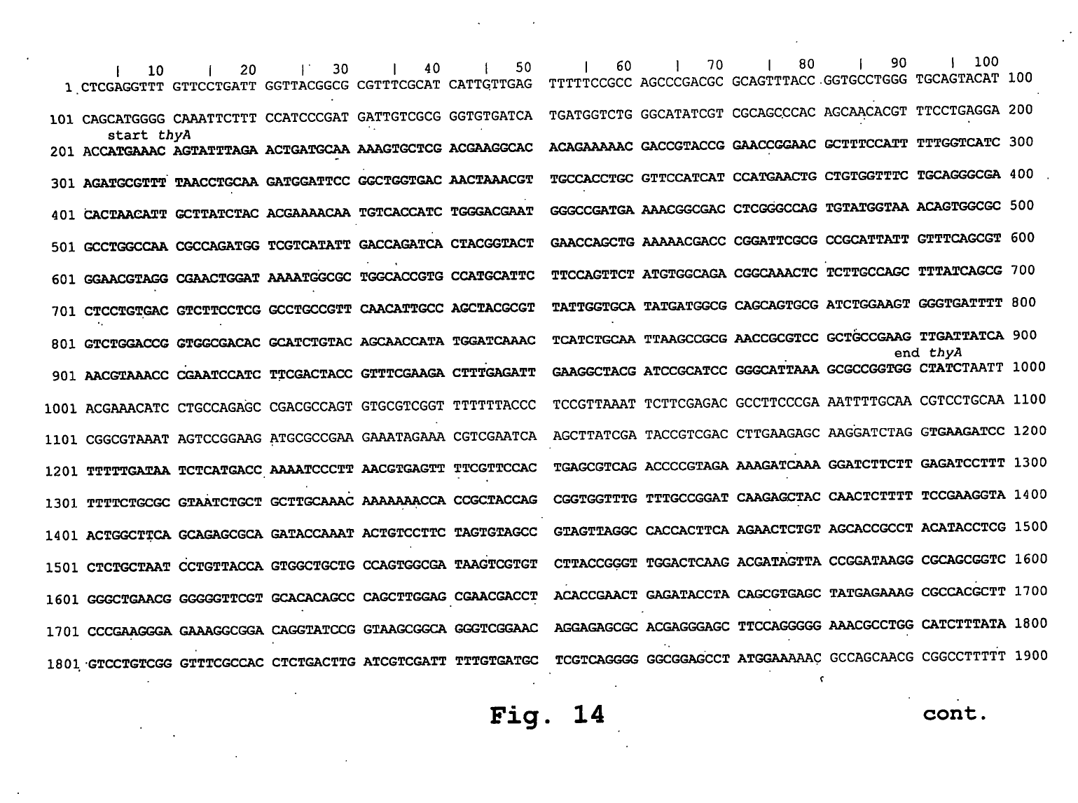

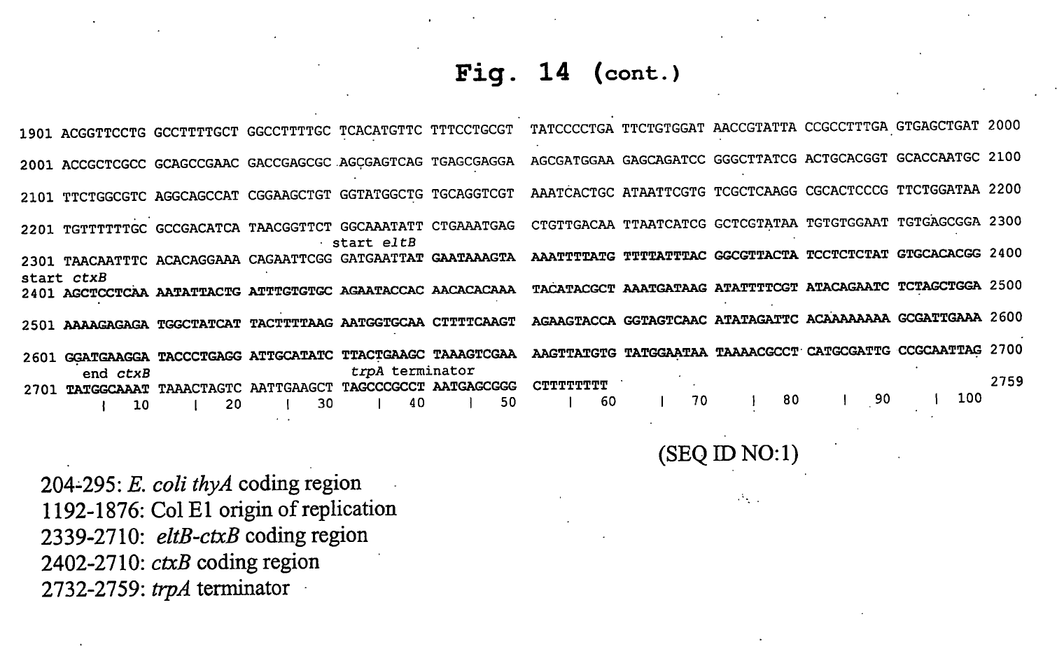

Plasmid DNA was purified from CsCl gradient ultracentrifugation and sequenced The complete nucleotide sequence of plasmid pMT-ctxBthyA-2 is given in FIG. 14. 95% of the plasmid has been sequenced (to be completed) at the stage before production of seed lots. In the Master Working Seed lot the coding region for the ctxB gene has been sequenced from two different tubes in the Master Seed lot Bank (as indicated in FIG. 13). End of production cells from three consistency lots has also been sequenced. The sequence obtained from both Master Seed lot as well as from consistency lots show 100% identity.

C.2. Amino-Terminal Sequence of the Mature Recombinant Protein.

In FIG. 15 the amino acid sequence from the rCTB produced by strain V. cholerae 401 is compared with the amino acid sequences of native CTB and native LTB toxin from E. coli. As can be seen in FIG. 15, the amino acid sequence of the signal peptide of LTB and rCTB 401 are identical as is the N-terminal amino acid of the mature protein. The comparison of native CTB (classical biotype) and rCTB 401 shows that the only difference is the N-terminal amino acid (Threonine in native CTB and Alanine in rCTB 401. This modification is justified by the previous experience with the rCTB 213 molecule [9], which also has the eltB signal sequence linked to the ctxB sequence. There were also four additional amino acids included in the sequence linkage region of this rCTB 213 molecule due to the methods for recombinant DNA technology available at the time [9].

Experience with the rCTB 213 in fermentor scale production revealed that up to six rCTB species with different amino termini could be isolated.

With this knowledge in mind the rCTB 401 linkage was designed so that the extra amino acids in the linkage region were removed plus that the N-terminal amino acid was replaced so as to be identical to that of native LTB i.e. an Alanine.

This modification has proven itself to be advantageous. There is only one N-termini in rCTB 401 isolated in all experimental and consistency batches of rCTB 401 irrespective of fermentation time and conditions.

C.3. Mode of Expression.

The pMT-ctxBthyA-2 plasmid that harbours the ctxB gene does not contain the gene for the strong repressor (lacI^{q}). In V. cholerae it is not known if there is a repressor (lacI) in the genome. V. cholerae do not ferment lactose but have a lacZ gene [12]. It is reasonable to assume that the eventual repressor will not be as potent as lacI^{q} and also that it would be present in much smaller quantities than the promotor (tacP) which is located on a high copy number plasmid. As a result the expression of agctb is in practice constitutive.

D. Stability of the Expression System.

D.1. Storage Stability.

The Master and Working Seed lots of the V. cholerae strain 401 have been stored for less than a year respectively at −65° C. or colder. Genetic stability for both the Master and Working Seed lots have been demonstrated after 6 months of storage since 100% of the colonies produced rCTB when grown for production of consistency lots. Previous experience with the Seed lot system of the rCTB producing strain V. cholerae 213 indicate that for that strain stability is excellent for more than 7 years. The Master and Working Seed lots are included in a stability testing program with testing every 5 years.

D.2. Stability in Extended Culture Time.

In experiments designed to investigate the stability of the plasmid retention and rCTB production, the V. cholerae strain 401 has been grown on a shaker in Modified Syncase broth at 37° C. Every day the culture was diluted down 10.000 fold in fresh medium. This was commenced for 11 days. On day 7 and 11 the culture was spread on Modified Syncase agar and the ability of the colonies to produce rCTB was tested with a colony blotting technique [SBL Vaccin test method PT00020 as described below] using a monoclonal antibody specific for LTB and crossreacting with CTB. After more than 100 generations (11 days of growth) 100% of the colonies retained their capacity to produce rCTB.

SBL Test Method PT00020

As indicated above (see for example section B.1.7), PT00020 is a method which is used to distinguish colonies of the rCTB producing strain that can produce rCTB from those that have lost that capacity. The methodology used consists of growing the bacterial colonies to be tested on an agar plate, transferring the colonies to a nitrocellulose filter. This filter is incubated with a monoclonal antibody specific for pentameric rCTB, washed and then incubated with an anti mouse IgG alkaline phosphatase conjugate. After washing the filters are developed with a precipitating dye, leaving the rCTB colonies bluish-black while non-producing colonies are left essentially colourless

D.3. Production Stability.

The production scale for the V. cholerae strain 401 is 500 litres. The medium is the same modified syncase medium as used above with the exception that glucose is used instead of sucrose. To investigate the plasmid retention, and to show consistency, samples were taken at break-point from three consecutive 500 litre production fermentations. After approx. 18 hours of growth at 37° C. in the main 500 litre fermentor 100% of the cells have retained their capability of producing rCTB.

D.4. Stability of the Genetic Construct During Production Fermentation.

To show the stability of the genetic construct, DNA was prepared from breakpoint harvests of V. cholerae strain 401. The plasmid DNA was purified by CsCl ultracentrifugation and sequenced as outlined in FIG. 13. The first base in the consensus sequence corresponds to base No 2210 in pMT-ctxBthyA-2 DNA, the last base corresponds to base 220 in pMT-ctxBthyA-2. The sequenced region encompasses sequence before the tac promotor, the entire eltB-ctxB and ends 18 bases inside the coding region for the thyA gene. The sequence determined from samples taken during production fermentation show the identical DNA sequence of the tac promotor, the eltb-ctxB gene and flanking DNA as that obtained from seed lot, thereby demonstrating the stability of the construct.

### Comparative Examples I

“401” Strain vs “213” Strain

213 Production System

The rCTB component which is produced in a prior art 213 V. cholerae expression system is summarized as follows:

A ctxA deleted V. cholerae O1 (JS1569) was transfected with a plasmid (designated pJS752-3) containing the gene sequence for CTB under the control of a heterologous promoter, wherein the CTB coding sequence is linked to a sequence encoding a heterologous leader polypeptide (the E. coli LT leader sequence) to facilitate secretion of CTB from the host cell. The pJS752-3 plasmid was prepared by excision of the CTB gene in plasmid JS162 and inserting the gene into a plasmid vector PKK223-1 which contains the tacP promoter but not the lacIq gene present in PJS162 that is responsible for IPTG dependence (more details on the methods of preparing and using these plasmids are described herein and are provided in Sanchez and Holmgren (1989) ibid and U.S. Pat. Nos. 5,268,276, 5,823,4246 and 6,043,057 and EP Patent No 0368819B).

The pJS752-3 plasmid further comprises an antibiotic selectable marker (ampicillin resistance marker) to enable selection of suitable plasmids containing the CTB sequence. The designation of the V. cholerae production strain (JS1569) with the expression vector (pJS752-3) is the V. cholerae 213 strain.

The CTB was overexpressed and secreted from the 213 production strain in monomeric form, whereafter it assembles into the characteristic pentameric ring-like structure to provide rCTB having a molecular weight of approx 58 kDa. In this way, the rCTB consists only of the non-toxic part of the cholera enterotoxin (since the toxic A subunit has been genetically deleted from the production strain) but retains its ability to bind GM-1 receptors on the surface of intestinal epithelial cells (see U.S. Pat. Nos. 5,268,276, 5,823,4246 and 6,043,057, EP Patent No 0368819 and Sanchez et al (1989) (ibid) for more details on this expression system).

“401” Expression System

As described herein, a derivative of the JS1569 V. cholerae production strain which lacks the functionality of a thyA gene has been produced (for example, the thyA gene may be removed or may be genetically disabled). A functional thyA gene is provided in an expression plasmid which allows for the selection of V. cholerae host cells which retain the plasmid and which are unable to grow in the absence of thymine (as described in WO 99/61634). The designation of the derived V. cholerae production strain (JS1569 ΔthyAΔkan) with the expression vector (pMT-CtxBthyA-2) as described herein is the CTB producing 401 V. cholerae strain.

TABLE 4
Comparison of yield of rCTB-401 and rCTB-213 in three consecutive fermentations

| | rCTB-401 in year 1998 | | | rCTB-213 in year 1999 | | | Average yield of |
|---|---|---|---|---|---|---|---|
| | Batch nr | | | | | | rCTB-213 in 1999-2001 |
| | M4806 | M4807 | M4808 | RC2902 | RC2903 | RC2904 | (about 60 batches) |
| mg rCTB/ml | 1.2 | 1.4 | 1.5 | 0.44 | 0.48 | 0.52 | 0.41 ± 0.07 |
| | | | | | | | (±1 SD) |

The data in Table 4 demonstrates that the average yield of rCTB of about 1.4 mg/ml from the thyA deleted V. cholerae strain (termed the “401” strain) at the end of the fermentation period is in the range of 3-4 times greater than the yield of rCTB (0.4 mg/ml) from the “213” strain).

The method used for measuring the rCTB concentration is single radial immunodiffusion (SRI) also known as the Mancini test (Mancini et al. (1965) Immunochem 2: 235-254: Immunochemical quantitation of antigen by single radial immunodiffusion) using antisera against highly purified rCTB. The rCTB standard used for preparing a calibration curve is a highly purified rCTB which has been characterised by a number of protein tests.

This yield (1.4 mg/ml) is about 50 fold higher than that reported from wild type V. cholerae 569B strain and about 20-fold higher than that reported by Sanchez and Holmgren (1989).

Discussion of Comparative Examples I

The main differences between the expression plasmids used in the prior art “213” production system and the “401” production system are outlined in Table 3. These include the absence of an antibiotic resistance marker, a smaller plasmid size and a higher yield of rCTB from the “401” strain relative to the “213” strain.

Without wishing to be bound by theory, it is believed that removing a portion of non-coding V. cholerae DNA downstream of the ctxB gene resulting in the reduced size of the expression vector contributes to the improved stability and the improved yield of the CTB end product. By way of example of the improved plasmid stability, the plasmid containing the cassette was still present in 100% of the bacterial cells in a culture after 100 generations even in the absence of antibiotic selection. The absence of an antibiotic resistance marker in the “401” strain also has advantages in terms of a safer and cheaper CTB end product. The produced rCTB is also advantageous because a more homogeneous CTB product is produced. In this respect, when the production strain is the 401 strain, only one rCTB is produced and this rCTB sequence differs from the wild type CTB sequence only in a single N terminal mutation (substitution of Threonine (Thr) to Alanine (Ala)). In contrast, when the production strain is the 213 strain, the final rCTB product actually contains slightly different rCTB amino acid sequences (see Sanchez and Holmgren 1989 (ibid)) as there are at least two different mutations occurring within the N-terminal residues of the CTB sequence.

### Comparative Examples II

Levels of CTB Produced Using 358 Strain (Lebens) and 401 Expression System

The main differences between the expression plasmids used in the Lebens et al (1993) (ibid) production system and the “401” production system are outlined in Table 3. These include the absence of an antibiotic resistance marker and a smaller plasmid size.

The CTB expression system described in Lebens et al (1993) requires the presence of an antibiotic whenever the organism is grown. The antibiotic resistance marker is an ampicillin resistance marker. The ampicillin resistance is due to the expression of the enzyme β-lactamase which cleaves the antibiotic. The V. cholerae strain termed the “358” strain used in the Lebens expression system requires the continuous presence of ampicillin in the medium in order to maintain optimum production. Thus, the CTB yields obtained using the Lebens expression system are only obtained using “selective pressure and in the presence of ampicillin”.

Levels of CTB Produced Using the Expression System Disclosed in WO 01/127144 and the “401” Expression System

Production of Recombinant CTB in Bacteria

The expression plasmid MS-0 (see FIG. 2 of WO 01/27144) was used to express rCTB and variants thereof. MS-0 containing the rCTB gene is named pML-CTBtacl. The plasmid pML-CTBtacl surprisingly generates up to five times the product which was generated by a comparable plasmid (Vector pJS162 as disclosed in Sanchez and Holmgren 1989 ibid). pML-CTBtacl was constructed by cloning a portion of the CTB genomic region and the complete CTB coding region into plasmid MS-0 creating a 3.66 Kb expression plasmid. The PvuII site in the polylinker was destroyed during cloning. The plasmid contains a tac promoter from pKK223, an EcoRI-BamHI polylinker fragment, and can be found at genbank accession No M77749.

The encoded protein is identical to the sequence from V. cholera strain 569B (SEQ ID NO: 2). The signal sequence (SEQ ID NO: 3) is also from the CTB V. cholera classic strain 569B CTB gene. The complete nucleotide sequence of V. cholera strain 569B CTB gene is shown in FIG. 1 (SEQ ID NO: 1) of WO 01/27144. The signal sequence for LTB (SEQ ID NO: 13) is MNKVKCYVLFTALLSSLCAYG is also shown in the sequence listing of WO 01/27144 and can be used in the production of mutants or variants of LTB.

Comparison with the 401 Expression System

The main differences between the expression plasmid used in the CTB production system disclosed in WO 01/27144 and the “401” production system are outlined in Table 3. These include the absence of an antibiotic resistance marker, a smaller plasmid size and a higher yield of rCTB from the “401” strain relative to the yields obtained from the expression system disclosed in WO 01/27144.

Overall Summary

It is well known that high level transcription and translation of proteins depends on many factors. These factors include but are not limited to: promoter strength, translational initiation sequences, codon choice, secondary structure of mRNA, transcriptional termination, plasmid copy number, plasmid stability and host cell physiology. Thus, the expression of different proteins can vary dramatically and the use of a strong promoter alone does not guarantee the successful overexpression of a desired protein.

This present invention teaches how to improve CTB yields using a CTB production system with markers other than antibiotic resistance markers and appropriate host cell strains that remove the need for antibiotic selection. The CTB production system comprises a bacterial host cell lacking the functionality of a thyA gene which is used in conjunction with a novel expression vector to produce unexpected high yields of recombinant B subunit of the cholera toxin (rCTB) relative to the yields obtained with known bacterial host cell production systems.

In one embodiment, the present invention teaches how to improve CTB yields using a CTB production system comprising a V. cholerae host cell lacking the functionality of a thyA gene which is used in conjunction with a novel expression vector to produce unexpected high yields of recombinant B subunit of the cholera toxin (rCTB) relative to the yields obtained with known V. cholerae production systems.

A plasmid expression vector was constructed in which the thymidylate synthase (thyA) gene of E. coli was used as a means of selection and maintenance of a plasmid comprising a CTB gene. The plasmids is of reduced size relative to known expression plasmids for producing CTB because substantially all of the non coding V. cholerae DNA downstream of the CTB gene was removed.

The unexpected high yield of CTB obtained using this expression system demonstrated both the efficiency of expression of heterologous genes in the V. cholerae strain and the stability of the plasmids maintained by complementation of the thyA deletion. Furthermore, the plasmid was found to be extremely stable. Even after repeated passages through liquid culture equivalent to 100 generations all the cells retained the plasmid and the ability to express the recombinant protein.

The expression system as reported here is advantageous because it facilitates the production of CTB for the following uses which include but are not limited to:

a protective immunogen in oral vaccination against cholera and LT-caused E. coli diarrhoea;

An immunomodulator or a tolerogenic inducing agent or an immune-deviating agent for down-regulating/modulating/de-sensitising/re-directing the immune response;

An adjuvant for altering, enhancing, directing, re-directing, potentiating or initiating an antigen-specific or non-specific immune response;

A carrier to stimulating an immune response to one or more unrelated antigens; and

A diagnostic agent for producing antibodies (such as monoclonal or polyclonal antibodies) for use in diagnostic or immunodiagnostic tests.

It is a particular advantage from the point of purification and standardisation of CTB as a vaccine component that relatively high yields of CTB can be achieved using stable bacterial host cell strains that lack the functionality of a thyA gene.

The present invention also teaches how to obtain a stable CTB preparation which is essentially free of antibiotic residues resulting in a safer product for human use.

### SPIRIT AND SCOPE OF THE INVENTION

The present invention is not to be limited in scope by the specific embodiments described herein. Indeed, various modifications of the invention in addition to those described herein will become apparent to those skilled in the art from the foregoing description and the accompanying figures. Such modifications are intended to fall within the

It is further to be understood that all values are approximate, and are provided for description. Patents, patent applications, publications, product descriptions, and protocols are cited throughout this application, the disclosures of which are incorporated herein by reference in their entireties for all purposes. In the case of inconsistencies, the present disclosure, including definitions, will prevail.

### REFERENCES

1. Lebens, M., and J. Holmgren. 1994. Structure and arrangement of the cholera toxin genes in V. cholerae 0139. FEMS Microbiology Letters. 117:197-202.
2. Kaper, J. B., H. Lockman, M. M. Baldini, and M. M. Levine. 1984. A recombinant live oral cholera vaccine. Biotechnology 2:345-349.
3. Sandkvist, M., T. R. Hirst, and M. Bagdasarian. 1987. Alterations at the carboxyl terminus change assembly and secretion properties of the B subunit of Escherichia coli heat-labile enterotoxin. J. Bacteriol. 169:4570-4576.
4. Lebens, M., S. Johansson, J. Osek, M. Lindblad and J. Holmgren. 1993. Large scale production of V. cholerae toxin B subunit for use in oral vaccines. Biotechnology 11:1574-1578.
5. Miller, V. L., and J. J. Mekalanos. 1988. A novel suicide vector and its use in construction of insertion mutations: osmoregulation of outer membrane proteins and virulence determinants in V. cholerae requires toxR J. Bacteriol. 170:2575-2583.
6. Milton, D. L., A. Nordqvist and H. Wolf-Watz. 1992. Cloning of a Metalloprotease gene involved in the virulence mechanism of Vibrio anguillarum. J. Bacteriol. 174:7235-7244.
7. Milton, D. L., R. O'Toole, P. Hörstedt and H. Wolf-Watz. 1996. Flagellin A is essential for the virulence of Vibrio anguillarium. J. Bacteriol. 178:1310-1319.
8. Levine, M. M., J. B. Kaper, D. Herrington, J. Ketley, G. Losonsky, C. O. Tacket, B. Tall and S. Cryz. 1988. Safety, immunogenicity, and efficacy of recombinant live oral cholera vaccines, CVD 103 and CVD 103-HgR. Lancet ii:467-470.
9. Sanchez, J., and J. Holmgren. 1989. Recombinant system for overexpression of cholera toxin B subunit in V. cholerae as a basis for vaccine development. Proc. Natl. Acad. Sci. USA. 86:481-485.
12. Parsot, C. 1992. Identification of a lacZ gene in V. cholerae. Res. Microbiol. 143:27-36.
13. Lebens, M., and J. Holmgren. 1994. Structure and arrangement of the cholera toxin genes in V. cholerae 0139. FEMS Microbiology Letters. 117:197-202.
14. Sanchez, J., and J. Holmgren. 1989. Recombinant system for overexpression of cholera toxin B subunit in V. cholerae as a basis for vaccine development. Proc. Natl. Acad. Sci. USA. 86:481-485.
15. Kaper, J. B., H. Lockman, M. M. Baldini, and M. M. Levine. 1984. A recombinant live oral cholera vaccine. Biotechnology 2:345-349.
16. Sandkvist, M., T. R. Hirst, and M. Bagdasarian. 1987. Alterations at the carboxyl terminus change assembly and secretion properties of the B subunit of Escherichia coli heat-labile enterotoxin. J. Bacteriol. 169:4570-4576.
17. Fürste, J. P., W. Pansegrau, R. Frank, H. Blöcker, P. Scholtz, M. Bagdasarian, and E. Lanka. 1986. Molecular cloning of the plasmid RP4 primase region in a multi-host-range tacP expression vector. Gene 48:119-131.
18. Sambrook, J., E. F. Fritsch, and T. Maniatis. 1989. Molecular cloning. A laboratory manual, 2nd ed. Cold Spring Harbor Laboratory Press, Cold Spring Harbor, N.Y.
19. Brosius, J., T. J. Dull, D. D. Sleeter, and H. F. Noller. 1981. Gene organization and primary structure of a ribosomal RNA operon from Escherichia coli. J. Mol. Biol. 148:107-127.
20. Gentz, R., A. Langner, A. C. Y. Chang, S. N. Cohen, and H. Bujard. 1981. Cloning and analysis of strong promoters is made possible by the downstream placement of a RNA termination signal. Proc. Natl. Acad. Sci. USA, 78:4936-4940.
21. Levine, M. M., J. B. Kaper, D. Herrington, J. Ketley, G. Losonsky, C. O. Tacket, B. Tall and S. Cryz. 1988. Safety, immunogenicity, and efficacy of recombinant live oral cholera vaccines, CVD 103 and CVD 103-HgR. Lancet ii:467-470.
22. Simon, R., U. Priefer, and A. Pühler. 1983. A broad host range mobilization system for in vivo genetic engineering; transposon mutagenesis in Gram negative bacteria. Biotechnology 1:784-791.
23. Turnbough, C. L., K. H. Kerr, W. R. Funderberg, J. P. Donahue and F. E. Powell. 1987. Nucleotide sequence and characterization of the pyrF operon of Escherichia coli K12. J. Biol. Chem. 262:10239-10245.
24. Theisen, M., R. A. Kelln and J. Neuhard. 1987. Cloning and characterization of the pyrF operon of Salmonella typhimurium. Eur. J. Biochem. 164:613-619.
25. Parsot, C. 1992. Identification of a lacZ gene in V. cholerae. Res. Microbiol. 143:27-36.

1. An expression system for producing a B subunit of a cholera toxin (CTB) wherein the expression system comprises:
(a) a Vibrio cholerae host cell lacking the functionality of a thyA gene; and
(b) an expression vector less than 5 kb in size comprising a functional thyA gene and a CTB gene which is substantially free of the flanking sequences immediately contiguous by the 5′ and 3′ end of the CTB gene in the naturally occurring genome of the host cell from which the CTB gene is derived.

2. The expression system according to claim 1 wherein the host cell lacks the functionality of a CTA gene.

3. The expression system according to claim 1 wherein the expression vector is about 3 kb in size.

4. The expression system according to any one of claims 1 wherein the expression vector comprises an E. coli thyA gene.

5. The expression system according to any one of claims 1 wherein the expression vector has the nucleotide sequence presented in SEQ ID NO:1.

6. The expression system according to any one of claims 1 wherein the expression vector further comprises at least one further nucleotide sequence encoding a heterologous protein.

7. The expression system according to claim 6 wherein the further nucleotide sequence encodes a non-toxic component or form of the heat labile E. coli enterotoxin LT, preferably the non-toxic component of LT is the B subunit of a (LTB) or a fragment thereof.

8. A method of producing CTB wherein the method comprises:
transforming a Vibrio cholerae host cell lacking the functionality of a thyA gene with an expression vector less than 5 kb in size comprising a functional thyA gene and a CTB gene which is substantially free of the flanking sequences immediately contiguous by the 5′ and 3′ end of the CTB gene in the naturally occurring genome of the host cell from which the CTB gene is derived,
and
culturing the transformed V. cholerae host cell under conditions which permit production of the CTB.

9. The method of claim 8 wherein the method further comprises isolating and/or purifying the CTB from the host cell.

10. An isolated nucleic acid construct which comprises a thyA gene and a CTB gene which is substantially free of the flanking sequences immediately contiguous by the 5′ and 3′ end of the CTB gene in the naturally occurring genome of the host cell from which the CTB gene is derived, and which nucleic acid construct is less than 5 kb in size.

11. The nucleic acid construct according to claim 10 wherein the nucleic acid construct is about 3 kb in size.

12. The nucleic acid construct according to claim 10, wherein the nucleic acid construct is a plasmid.

13. The nucleic acid construct according to claim 12, wherein the plasmid is pMT-ctxBthyA-2 characterised by a restriction endonuclease map as shown in FIG. 13.

14. The nucleic acid construct according to claim 12, wherein the plasmid has the nucleotide sequence SEQ ID NO: 1.
